SEQUENCE LISTING (20 sequences)

SEQ ID NO: 1 - protein, 110 aa, synthetic construct
  REGION 1..110: note = Synthetic: Clone 5C1 (intracellular) VH
QSLEESRGGLFKPTDTLTLTCTVSGFSLKTYVINWVRQAPGNGLEWIGIIDTGDMTYYASWAKSRSTITRNTNENTVTLKMTSLTAADTATYFCASGDIWGPGTLVTVSS

SEQ ID NO: 2 - protein, 112 aa, synthetic construct
  REGION 1..112: note = Synthetic: Clone 5C1 (intracellular) VL
AQVLTQTASPVSAAVGGTVTINCQASQSVYSNWLSWFQQKPGQPPKRLIYSASTLASGVSSRFKGSGSGTQFTLTISDVQCDDAATYYCLGEFNCARADCRAFGGGTEVVVK

SEQ ID NO: 3 - protein, 113 aa, synthetic construct
  REGION 1..113: note = Synthetic: Clone 9H5 (Extracellular) VH
QSLEESGGRLVTPGGSLTLTCTVSGFSLSAYAMSWVRQAPGKGLEWIGYINYDGIAYYTNWAKGRFTISKTSTTVDLKITSPTTEDTAAYFCASDLLGVFNLWGQGTLVTVSS

SEQ ID NO: 4 - protein, 111 aa, synthetic construct
  REGION 1..111: note = Synthetic: Clone 9H5 (Extracellular) VL
AAVLTQTASPVSAAVGGTVTINCQASQSVYSNNRLSWFQQKPGQPPKLLIYYASFLASGVPSRFKGSGSGAQFTLTISDLQCDDAATYYCAGGYISYSDNAFGGGTEVVVK

SEQ ID NO: 5 - protein, 114 aa, synthetic construct
  REGION 1..114: note = Synthetic: Clone 10A2 (Extracellular) VH
QSVEESGGRLVTPGTPLTLTCTASGFSLTTYAMGWVRQAPGKGLEWIGYIWSSGTTDYASWAKGRFTISKTSPTVDLKMTSPTTEDTATYFCARYINYVTGDLWGQGTLVTVSS

SEQ ID NO: 6 - protein, 113 aa, synthetic construct
  REGION 1..113: note = Synthetic: Clone 10A2 VL
AQVLTQTPSPVSADVGGTVTINCQASQSVYNNVLLAWYQQKAGQPPKLLIYKASTLASGVSSRFKGSGSGTQFTLTISGVQCDDAATYYCQGEFSCSSADCTAFGGGTEVVVK

SEQ ID NO: 7 - protein, 104 aa, synthetic construct
  REGION 1..104: note = Synthetic: Constant (Kappa1)
GDPVAPTVLIFPPAADQVATGTVTIVCVANKYFPDVTVTWEVDGTTQTTGIENSKTPQNSADCTYNLSSTLTLTSTQYNSHKEYTCKVTQGTTSVVQSFNRGDC

SEQ ID NO: 8 - protein, 323 aa, synthetic construct
  REGION 1..323: note = Synthetic: VH (IgG)
GQPKAPSVFPLAPCCGDTPSSTVTLGCLVKGYLPEPVTVTWNSGTLTNGVRTFPSVRQSSGLYSLSSVVSVTSSSQPVTCNVAHPATNTKVDKTVAPSTCSKPTCPPPELLGGPSVFIFPPKPKDTLMISRTPEVTCVVVDVSQDDPEVQFTWYINNEQVRTARPPLREQQFNSTIRVVSTLPIAHQDWLRGKEFKCKVHNKALPAPIEKTISKARGQPLEPKVYTMGPPREELSSRSVSLTCMINGFYPSDISVEWEKNGKAEDNYKTTPAVLDSDGSYFLYSKLSVPTSEWQRGDVFTCSVMHEALHNHYTQKSISRSPGK

SEQ ID NO: 9 - protein, 19 aa, synthetic construct
  REGION 1..19: note = Synthetic: amino terminal signal peptide
METGLRWLLLVAVLKGVQC

SEQ ID NO: 10 - protein, 22 aa, synthetic construct
  REGION 1..22: note = Synthetic: amino terminal signal peptide
MDTRAPTQLLGLLLLWLPGATF

SEQ ID NO: 11 - protein, 19 aa, synthetic construct
  REGION 1..19: note = Synthetic: amino terminal signal peptide
METGLRWLLLVAVLKGVQC

SEQ ID NO: 12 - protein, 22 aa, synthetic construct
  REGION 1..22: note = Synthetic: amino terminal signal peptide
MDTRAPTQLLGLLLLWLPGAAF

SEQ ID NO: 13 - protein, 19 aa, synthetic construct
  REGION 1..19: note = Synthetic: amino terminal signal peptide
METGLRWLLLVAVLKGVQC

SEQ ID NO: 14 - protein, 22 aa, synthetic construct
  REGION 1..22: note = Synthetic: amino terminal signal peptide
MDTRAPTQLLGLLLLWLPGATF

SEQ ID NO: 15 - protein, 452 aa, synthetic construct
  REGION 1..452: note = Synthetic: Clone 5C1 (intracellular) Full length heavy chain, including signal peptide
METGLRWLLLVAVLKGVQCQSLEESRGGLFKPTDTLTLTCTVSGFSLKTYVINWVRQAPGNGLEWIGIIDTGDMTYYASWAKSRSTITRNTNENTVTLKMTSLTAADTATYFCASGDIWGPGTLVTVSSGQPKAPSVFPLAPCCGDTPSSTVTLGCLVKGYLPEPVTVTWNSGTLTNGVRTFPSVRQSSGLYSLSSVVSVTSSSQPVTCNVAHPATNTKVDKTVAPSTCSKPTCPPPELLGGPSVFIFPPKPKDTLMISRTPEVTCVVVDVSQDDPEVQFTWYINNEQVRTARPPLREQQFNSTIRVVSTLPIAHQDWLRGKEFKCKVHNKALPAPIEKTISKARGQPLEPKVYTMGPPREELSSRSVSLTCMINGFYPSDISVEWEKNGKAEDNYKTTPAVLDSDGSYFLYSKLSVPTSEWQRGDVFTCSVMHEALHNHYTQKSISRSPGK

SEQ ID NO: 16 - protein, 238 aa, synthetic construct
  REGION 1..238: note = Synthetic: Clone 5C1 Full length light chain, including signal peptide
MDTRAPTQLLGLLLLWLPGATFAQVLTQTASPVSAAVGGTVTINCQASQSVYSNWLSWFQQKPGQPPKRLIYSASTLASGVSSRFKGSGSGTQFTLTISDVQCDDAATYYCLGEFNCARADCRAFGGGTEVVVKGDPVAPTVLIFPPAADQVATGTVTIVCVANKYFPDVTVTWEVDGTTQTTGIENSKTPQNSADCTYNLSSTLTLTSTQYNSHKEYTCKVTQGTTSVVQSFNRGDC

SEQ ID NO: 17 - protein, 455 aa, synthetic construct
  REGION 1..455: note = Synthetic: Clone 9H5 (Extracellular) Full length heavy chain, including signal peptide
METGLRWLLLVAVLKGVQCQSLEESGGRLVTPGGSLTLTCTVSGFSLSAYAMSWVRQAPGKGLEWIGYINYDGIAYYTNWAKGRFTISKTSTTVDLKITSPTTEDTAAYFCASDLLGVFNLWGQGTLVTVSSGQPKAPSVFPLAPCCGDTPSSTVTLGCLVKGYLPEPVTVTWNSGTLTNGVRTFPSVRQSSGLYSLSSVVSVTSSSQPVTCNVAHPATNTKVDKTVAPSTCSKPTCPPPELLGGPSVFIFPPKPKDTLMISRTPEVTCVVVDVSQDDPEVQFTWYINNEQVRTARPPLREQQFNSTIRVVSTLPIAHQDWLRGKEFKCKVHNKALPAPIEKTISKARGQPLEPKVYTMGPPREELSSRSVSLTCMINGFYPSDISVEWEKNGKAEDNYKTTPAVLDSDGSYFLYSKLSVPTSEWQRGDVFTCSVMHEALHNHYTQKSISRSPGK

SEQ ID NO: 18 - protein, 237 aa, synthetic construct
  REGION 1..237: note = Synthetic: Clone 9H5 (Extracellular) Full length light chain, including signal peptide
MDTRAPTQLLGLLLLWLPGAAFAAVLTQTASPVSAAVGGTVTINCQASQSVYSNNRLSWFQQKPGQPPKLLIYYASFLASGVPSRFKGSGSGAQFTLTISDLQCDDAATYYCAGGYISYSDNAFGGGTEVVVKGDPVAPTVLIFPPAADQVATGTVTIVCVANKYFPDVTVTWEVDGTTQTTGIENSKTPQNSADCTYNLSSTLTLTSTQYNSHKEYTCKVTQGTTSVVQSFNRGDC

SEQ ID NO: 19 - protein, 456 aa, synthetic construct
  REGION 1..456: note = Synthetic: Clone 10A2 Full length heavy chain, including signal peptide
METGLRWLLLVAVLKGVQCQSVEESGGRLVTPGTPLTLTCTASGFSLTTYAMGWVRQAPGKGLEWIGYIWSSGTTDYASWAKGRFTISKTSPTVDLKMTSPTTEDTATYFCARYINYVTGDLWGQGTLVTVSSGQPKAPSVFPLAPCCGDTPSSTVTLGCLVKGYLPEPVTVTWNSGTLTNGVRTFPSVRQSSGLYSLSSVVSVTSSSQPVTCNVAHPATNTKVDKTVAPSTCSKPTCPPPELLGGPSVFIFPPKPKDTLMISRTPEVTCVVVDVSQDDPEVQFTWYINNEQVRTARPPLREQQFNSTIRVVSTLPIAHQDWLRGKEFKCKVHNKALPAPIEKTISKARGQPLEPKVYTMGPPREELSSRSVSLTCMINGFYPSDISVEWEKNGKAEDNYKTTPAVLDSDGSYFLYSKLSVPTSEWQRGDVFTCSVMHEALHNHYTQKSISRSPGK

SEQ ID NO: 20 - protein, 239 aa, synthetic construct
  REGION 1..239: note = Synthetic: Clone 10A2 Full length light chain, including signal peptide
MDTRAPTQLLGLLLLWLPGATFAQVLTQTPSPVSADVGGTVTINCQASQSVYNNVLLAWYQQKAGQPPKLLIYKASTLASGVSSRFKGSGSGTQFTLTISGVQCDDAATYYCQGEFSCSSADCTAFGGGTEVVVKGDPVAPTVLIFPPAADQVATGTVTIVCVANKYFPDVTVTWEVDGTTQTTGIENSKTPQNSADCTYNLSSTLTLTSTQYNSHKEYTCKVTQGTTSVVQSFNRGDC

(19) United States
(12) Patent Application Publication
(10) Pub. No.: US 2024/0059782 A1
(43) Pub. Date: Feb. 22, 2024

(54) ANTI-HUMAN B CELL MATURATION ANTIGEN (BCMA) ANTIBODIES AND THEIR USE IN IMMUNOHISTOCHEMISTRY (IHC)

(71) Applicant: Agilent Technologies, Inc., Santa Clara, CA (US)

(72) Inventors: Morten Draeby SORENSEN, Herlev (DK); Tine HAGEDORN-OLSEN, Skibby (DK); Kirsten HOFF, Maaloev (DK)

(21) Appl. No.: 18/116,008

(22) Filed: Mar. 1, 2023

Related U.S. Application Data
(60) Provisional application No. 63/315,297, filed on Mar. 1, 2022.

(51) Int. Cl. C07K 16/28; G01N 33/574

(52) U.S. Cl. CPC C07K 16/2878; G01N 33/57492; G01N 2333/70578

(57) ABSTRACT

In alternative embodiments, provided are chimeric or recombinant anti-human B-cell maturation antigen (BCMA, or BCM) polypeptide (also called tumor necrosis factor receptor superfamily member 17 (TNERSF17)) antibodies, including products of manufacture and kits comprising them, and methods for making and using them, including for example their use in the detection or diagnosis of a cancer or other conditions. In alternative embodiments, anti-BCMA antibodies as provided herein are used together with an agent for determining whether BCMA expression or activity is reduced or absent.

### RELATED APPLICATIONS

This United States (U.S.) utility patent application claims the benefit of priority under 35 U.S.C. § 119(e) to U.S. Provisional Patent Application Serial No. (USSN) 63/315,297, Mar. 1, 2022. The aforementioned application is expressly incorporated herein by reference in its entirety and for all purposes.

### TECHNICAL FIELD

This invention generally relates to immunohistochemistry (IHC) and cancer diagnosis. In alternative embodiments, provided are chimeric or recombinant anti-human B-cell maturation antigen (BCMA, or BCM) polypeptide (also called tumor necrosis factor receptor superfamily member 17 (TNFRSF17)) antibodies, including products of manufacture and kits comprising them, and methods for making and using them, including for example their use in the detection or diagnosis of a cancer or other conditions. In alternative embodiments, anti-BCMA antibodies as provided herein are used together with an agent for determining whether BCMA expression or activity is reduced or absent.

### BACKGROUND

B-cell maturation antigen (BCMA or BCM), also known as tumor necrosis factor receptor superfamily member 17 (TNFRSF17), is a type III transmembrane glycoprotein, a spanning protein receptor that in humans is encoded by the TNFRSF17 gene. BCMA is a cell surface receptor of the TNF receptor superfamily which recognizes B-cell activating factor (BAFF).

BCMA is a receptor for B-cell activation factor (BAFF) and a proliferation inducing ligand (APRIL)^{1, 2}. BCMA is induced in late memory B-cells committed to the plasma cell (PC) differentiation and is expressed on the surface of plasmablasts, germinal centre cells and differentiated plasma cells. BCMA is not expressed on other cells from human organs but is expressed on plasma cells (PCs) from Multiple Myeloma (MM) patients^{1, 2}.

The biological function of BCMA in normal physiology, is to support survival of long-lived PCs, production of antibodies and class switch of immunoglobulin. In the tumour environment BCMA promote proliferation and survival of MM cells, BCMA is associated with immunosuppressive Bone Marrow (BM) microenvironment and increased serum BCMA content is associated with disease progression and poorer outcome³.

BCMA is expressed on Multiple Myeloma (MM) cells and is a potential target for immunotherapy in MM^{1, 2, 4, 5}, and in B-cell lymphoma⁶. Anti-BCMA targeted immunotherapy has proven promising in MM patients, using Chimeric Antigen Receptor (CAR)T-cells^{2, 3, 5}, antibody-drug conjugates (ADCs), bispecific T-cell engager (BiTE), bispecific molecule and bi/trispecific Abs^{3, 5, 6}. Apart from MM, BCMA is also expressed in lymphoma samples including Hodgkin (HL), Non-Hodgkin (NHL), Diffuse Large B-cell Lymphoma (DLBCL), Marginal cell Lymphoma (MCL), Follicular Lymphoma (FL) and Marginal Zone Lymphoma (MZL). Data supports further development of anti BCMA CAR T-cells as a potential treatment for not only MM but also some lymphomas⁴.

BCMA is a challenging target to develop antibodies against due to the small and complex nature of this membrane spanning protein.

### SUMMARY

In alternative embodiments, provided are isolated, recombinant or purified antibodies (Abs), or antigen (Ag) binding fragments thereof, or monomeric or dimeric antigen binding proteins (ABPs), capable of specifically binding a human B-cell maturation antigen (BCMA, or BCM) polypeptide (also called tumor necrosis factor receptor superfamily member 17 (TNFRSF17)),
wherein the isolated or purified Ab, or Ag binding fragment thereof, or monomeric or dimeric ABP comprises:
(a) a heavy chain variable region (VH) comprising:
   (1) an amino acid sequence comprising the three CDR1, CDR2 and CDR3 complementarity determining regions (CDRs) of SEQ ID NO:1, or CDR1 amino acid (aa) residues GFSLKTYV (residues 25-32 of SEQ ID NO:1), CDR2 aa residues IDTGDMT (residues 50 to 56 of SEQ ID NO:1), and CDR3 aa residues ASGDI (residues 95-99 of SEQ ID NO:1), or
   (2) amino acid sequences having at least about 70%, 75%, 80%, 85%, 90%, 95%, 98% sequence identity, or between about 70% to 100% sequence identity, to each of the three CDR1, CDR2 and CDR3 complementarity determining regions (CDRs) of SEQ ID NO:1, or CDR1 amino acid (aa) residues GFSLKTYV (residues 25-32 of SEQ ID NO:1), CDR2 aa residues IDTGDMT (residues 50 to 56 of SEQ ID NO:1), and CDR3 aa residues ASGDI (residues 95-99 of SEQ ID NO:1), or
   (3) an amino acid sequence having at least about 70%, 75%, 80%, 85%, 90%, 95%, 98% sequence identity, or between about 70% to 100% sequence identity, to SEQ ID NO:1, or an amino acid sequence having complete sequence identity to SEQ ID NO:1;
(b) a light chain variable region (VL) comprising:
   (1) an amino acid sequence comprising the three CDR1, CDR2 and CDR3 complementarity determining regions (CDRs) of SEQ ID NO:2, or CDR1 amino acid (aa) residues QSVYSNW (residues 27-33 of SEQ ID NO:2), CDR2 aa residues SAS (residues 51 to 53 of SEQ ID NO:2), and CDR3 aa residues LGEFNCARADCRA (residues 90-102 of SEQ ID NO:2), or
   (2) amino acid sequences having at least about 70%, 75%, 80%, 85%, 90%, 95%, 98% sequence identity, or between about 70% to 100% sequence identity, to each of the three CDR1, CDR2 and CDR3 complementarity determining regions (CDRs) of SEQ ID NO:2, or CDR1 amino acid (aa) residues QSVYSNW (residues 27-33 of SEQ ID NO:2), CDR2 aa residues SAS (residues 51 to 53 of SEQ ID NO:2), and CDR3 aa residues LGEFNCARADCRA (residues 90-102 of SEQ ID NO:2); or
   (3) an amino acid sequence having at least about 70%, 75%, 80%, 85%, 90%, 95%, 98% sequence identity, or between about 70% to 100% sequence identity, to SEQ ID NO:2, or an amino acid sequence having complete (100%) sequence identity to SEQ ID NO:2;
(c) the heavy chain variable region (VH) of (a) and the light chain variable region (VL) of (b);
(d) a heavy chain variable region (VH) comprising:
   (1) an amino acid sequence comprising the three CDR1, CDR2 and CDR3 complementarity determining regions (CDRs) of SEQ ID NO:3, or CDR1 amino acid (aa) residues GFSLSAYA (residues 25-32 of SEQ ID NO:3), CDR2 aa residues INYDGIA (residues 50 to 56 of SEQ ID NO:3), and CDR3 aa residues ASDLLGVFNL (residues 93-102 of SEQ ID NO:3), or
   (2) amino acid sequences having at least about 70%, 75%, 80%, 85%, 90%, 95%, 98% sequence identity, or between about 70% to 100% sequence identity, to each of the three CDR1, CDR2 and CDR3 complementarity determining regions (CDRs) of SEQ ID NO:3, or CDR1 amino acid (aa) residues GFSLSAYA (residues 25-32 of SEQ ID NO:3), CDR2 aa residues INYDGIA (residues 50 to 56 of SEQ ID NO:3), and CDR3 aa residues ASDLLGVFNL (residues 93-102 of SEQ ID NO:3), or
   (3) an amino acid sequence having at least about 70%, 75%, 80%, 85%, 90%, 95%, 98% sequence identity, or between about 70% to 100% sequence identity, to SEQ ID NO:3, or an amino acid sequence having complete sequence identity to SEQ ID NO:3;
(e) a light chain variable region (VL) comprising:
   (1) an amino acid sequence comprising the three CDR1, CDR2 and CDR3 complementarity determining regions (CDRs) of SEQ ID NO:4, or CDR1 amino acid (aa) residues QSVYSNNR (residues 27-34 of SEQ ID NO:4), CDR2 aa residues YAS (residues 52 to 55 of SEQ ID NO:4), and CDR3 aa residues AGGYISYSDNA (residues 91-101 of SEQ ID NO:4), or
   (2) amino acid sequences having at least about 70%, 75%, 80%, 85%, 90%, 95%, 98% sequence identity, to each of the three CDR1, CDR2 and CDR3 complementarity determining regions (CDRs) of SEQ ID NO:4, or CDR1 amino acid (aa) residues QSVYSNNR (residues 27-34 of SEQ ID NO:4), CDR2 aa residues YAS (residues 52 to 55 of SEQ ID NO:4), and CDR3 aa residues AGGYISYSDNA (residues 91-101 of SEQ ID NO:4); or
   (3) an amino acid sequence having at least about 70%, 75%, 80%, 85%, 90%, 95%, 98% sequence identity, or between about 70% to 100% sequence identity, to SEQ ID NO:4, or an amino acid sequence having complete (100%) sequence identity to SEQ ID NO:4;
(f) the heavy chain variable region (VH) of (d) and the light chain variable region (VL) of (e);
(g) a heavy chain variable region (VH) comprising:
   (1) an amino acid sequence comprising the three CDR1, CDR2 and CDR3 complementarity determining regions (CDRs) of SEQ ID NO:5, or CDR1 amino acid (aa) residues GFSLTTYA (residues 25-32 of SEQ ID NO:5), CDR2 aa residues IWSSGTT (residues 50 to 56 of SEQ ID NO:5), and CDR3 aa residues ARYINYVTGDL (residues 93-103 of SEQ ID NO:5), or
   (2) amino acid sequences having at least about 70%, 75%, 80%, 85%, 90%, 95%, 98% sequence identity, or between about 70% to 100% sequence identity, to each of the three CDR1, CDR2 and CDR3 complementarity determining regions (CDRs) of SEQ ID NO:5, or CDR1 amino acid (aa) residues GFSLTTYA (residues 25-32 of SEQ ID NO:5), CDR2 aa residues IWSSGTT (residues 50 to 56 of SEQ ID NO:5), and CDR3 aa residues ARYINYVTGDL (residues 93-103 of SEQ ID NO:5), or
   (3) an amino acid sequence having at least about 70%, 75%, 80%, 85%, 90%, 95%, 98% sequence identity, or between about 70% to 100% sequence identity, to SEQ ID NO:5, or an amino acid sequence having complete sequence identity to SEQ ID NO:5;
(h) a light chain variable region (VL) comprising:
   (1) an amino acid sequence comprising the three CDR1, CDR2 and CDR3 complementarity determining regions (CDRs) of SEQ ID NO:6, or CDR1 amino acid (aa) residues QSVYNNVL (residues 27-34 of SEQ ID NO:6), CDR2 aa residues KAS (residues 52 to 54 of SEQ ID NO:6), and CDR3 aa residues QGEFSCSSADCTA (residues 91-103 of SEQ ID NO:6), or
   (2) amino acid sequences having at least about 70%, 75%, 80%, 85%, 90%, 95%, 98% sequence identity, to each of the three CDR1, CDR2 and CDR3 complementarity determining regions (CDRs) of SEQ ID NO:6, or CDR1 amino acid (aa) residues QSVYNNVL (residues 27-34 of SEQ ID NO:6), CDR2 aa residues KAS (residues 52 to 54 of SEQ ID NO:6), and CDR3 aa residues QGEFSCSSADCTA (residues 91-103 of SEQ ID NO:6); or
   (3) an amino acid sequence having at least about 70%, 75%, 80%, 85%, 90%, 95%, 98% sequence identity, or between about 70% to 100% sequence identity, to SEQ ID NO:6, or an amino acid sequence having complete (100%) sequence identity to SEQ ID NO:6; and/or
(i) the heavy chain variable region (VH) of (g) and the light chain variable region (VL) of (h).

In alternative embodiments, the isolated or purified Ab, or Ag binding fragment thereof, or monomeric or dimeric ABP, is fabricated as or in the form of:
an antigen-binding fragment (Fab, or an Ab fragment having just one constant and one variable domain of each of an Ab heavy and light chain),
a F(ab′)₂ (or an Ab digested by pepsin yielding two fragments: a F(ab′)₂ fragment and a pFc′ (pepsin cleavage Fc) fragment),
a Fab′ (a single chain of a F(ab′)₂ fragment),
a single-chain variable fragment (scFv) (or a fusion protein of a variable region of an Ab heavy and light chain connected together with a linker peptide optionally of about ten to about 25 amino acids in length),
a (scFv)₂, or a di-scFv or a bi-scFv, or a single peptide chain having two variable heavy and two variable light regions yielding tandem scFv,
a minibody (or a fusion protein of a variable region of an Ab heavy and light chain connected together with an alkyl group, optionally a methyl or an ethyl group)
a diabody (or an scFv with a linker peptide too short (optionally about five amino acids) for the two variable regions to fold together forcing the scFvs to dimerize), a triabody or a tetrabody (or an scFv with a linker peptide too short (optionally about one or two amino acids) for the two variable regions to fold together forcing the scFvs to trimerize or tetramize),
a single-domain antibody (dAB) (or a single variable region of an Ab heavy or Ab light chain),
a plurality of complementarity determining region (CDR) fragments, or
a multi specific antibody formed from two or more antibody fragments.

In alternative embodiments of isolated, recombinant or purified antibodies (Abs), or antigen (Ag) binding fragments thereof, or monomeric or dimeric antigen binding proteins (ABPs), as provided herein:
the heavy chain variable region (VH), if present, comprises:
(a) an amino acid sequence:

(SEQ ID NO: 1)
QSLEESRGGLFKPTDTLTLTCTVSGFSLKTYVINWVRQAPGNGLEWIGI
IDTGDMTYYASWAKSRSTITRNTNENTVTLKMTSLTAADTATYFCASGD
IWGPGTLVTVSS,

or

SEQ ID NO:1 having one or more amino acid substitutions, additions (insertions) or deletions, and the recombinant Ab, or Ag binding fragment thereof, or monomeric or dimeric ABP retains its ability to specifically bind to a human BCMA protein or polypeptide;
(b) an amino acid sequence:

(SEQ ID NO: 3)
QSLEESGGRLVTPGGSLTLTCTVSGFSLSAYAMSWVRQAPGKGLEWIGY
INYDGIAYYTNWAKGRFTISKTSTTVDLKITSPTTEDTAAYFCASDLLG
VFNLWGQGTLVTVSS,

or

SEQ ID NO: 3. having one or more amino acid substitutions, additions (insertions) or deletions, and the recombinant Ab, or Ag binding fragment thereof, or monomeric or dimeric ABP retains its ability to specifically bind to a human BCMA protein or polypeptide; or
(c) an amino acid sequence:

(SEQ ID NO: 5)
QSVEESGGRLVTPGTPLTLTCTASGFSLTTYAMGWVRQAPGKGLEWIGY
IWSSGTTDYASWAKGRFTISKTSPTVDLKMTSPTTEDTATYFCARYINY
VTGDLWGQGTLVTVSS,

or

SEQ ID NO:5. having one or more amino acid substitutions, additions (insertions) or deletions, and the recombinant Ab, or Ag binding fragment thereof, or monomeric or dimeric ABP retains its ability to specifically bind to a human BCMA protein or polypeptide.

In alternative embodiments of isolated, recombinant or purified antibodies (Abs), or antigen (Ag) binding fragments thereof, or monomeric or dimeric antigen binding proteins (ABPs), as provided herein: the light chain variable region (VL), if present, comprises:
(a) an amino acid sequence:

(SEQ ID NO: 2)
AQVLTQTASPVSAAVGGTVTINCQASQSVYSNW
LSWFQQKPGQPPKRLIYSASTLASGVSSRFKGS
GSGTQFTLTISDVQCDDAATYYCLGEFNCARAD
CRAFGGGTEVVVK,

or

SEQ ID NO:2 having one or more amino acid substitutions, additions (insertions) or deletions, and the recombinant Ab, or Ag binding fragment thereof, or monomeric or dimeric ABP retains its ability to specifically bind to a human BCMA protein or polypeptide;
(b) an amino acid sequence:

(SEQ ID NO: 4)
AAVLTQTASPVSAAVGGTVTINCQASQSVYSNNRLSWFQQ
KPGQPPKLLIYYASFLASGVPSRFKGSGSGAQFTLTISDL
QCDDAATYYCAGGYISYSDNAFGGGTEVVVK,

or

SEQ ID NO:4 having one or more amino acid substitutions, additions (insertions) or deletions, and the recombinant Ab, or Ag binding fragment thereof, or monomeric or dimeric ABP, retains its ability to specifically bind to a human BCMA protein or polypeptide; or
(c) an amino acid sequence:

(SEQ ID NO: 6)
AQVLTQTPSPVSADVGGTVTINCQASQSVYNNVLLAWYQQ
KAGQPPKLLIYKASTLASGVSSRFKGSGSGTQFTLTISGV
QCDDAATYYCQGEFSCSSADCTAFGGGTEVVVK,

or

SEQ ID NO:6 having one or more amino acid substitutions, additions (insertions) or deletions, and the recombinant Ab, or Ag binding fragment thereof, or monomeric or dimeric ABP, retains its ability to specifically bind to a human BCMA protein or polypeptide.

In alternative embodiments of isolated, recombinant or purified antibodies (Abs), or antigen (Ag) binding fragments thereof, or monomeric or dimeric antigen binding proteins (ABPs), as provided herein:
the one or more amino acid substitutions comprise one or more conservative amino acid substitutions, and optionally the one or more amino acid substitutions comprise one or more conservative amino acid substitutions, and the isolated or purified Ab, or Ag binding fragment thereof, or monomeric or dimeric ABP, retains its ability to specifically bind to a human BCMA protein or polypeptide;
SEQ ID NO:1, SEQ ID NO:2, SEQ ID NO:3 or SEQ ID NO:4 has two, three, four, five, six, seven, eight, nine, ten, eleven, twelve thirteen, fourteen or fifteen conservative amino acid substitutions, and the recombinant Ab, or Ag binding fragment thereof, or monomeric or dimeric ABP retains its ability to specifically bind to a human BCMA protein or polypeptide;
the light chain variable region further comprises at least a portion of a light chain constant region, and optionally the light chain constant region comprises an amino acid sequence:

(SEQ ID NO: 7)
GDPVAPTVLIFPPAADQVATGTVTIVCVANKYFPDVTVTW
EVDGTTQTTGIENSKTPQNSADCTYNLSSTLTLTSTQYNS
HKEYTCKVTQGTTSVVQSFNRGDC;

the heavy chain variable region further comprises at least a portion of a heavy chain constant region, and optionally the heavy chain constant region comprises an amino acid sequence:

(SEQ ID NO: 8)
GQPKAPSVFPLAPCCGDTPSSTVTLGCLVKGYLPEPVTVT
WNSGTLINGVRTFPSVRQSSGLYSLSSVVSVTSSSQPVTC
NVAHPATNTKVDKTVAPSTCSKPTCPPPELLGGPSVFIFP
PKPKDTLMISRTPEVTCVVVDVSQDDPEVQFTWYINNEQV
RTARPPLREQQFNSTIRVVSTLPIAHQDWLRGKEFKCKV
HNKALPAPIEKTISKARGQPLEPKVYTMGPPREELSSRSV
SLTCMINGFYPSDISVEWEKNGKAEDNYKTTPAVLDSDGS
YFLYSKLSVPTSEWQRGDVFTCSVMHEALHNHYTQKSISR
SPGK;

the light chain variable region further comprises at least a portion of a light chain constant region; and, the heavy chain variable region further comprises at least a portion of a heavy chain constant region;
the heavy chain constant region comprises amino acid sequence from a IgG, IgM, IgA, IgD or IgE isotype;
the light chain constant region comprises amino acid sequence from a kappa (κ) or lambda (λ) isotype;
the at least a portion of the heavy chain constant region, at least a portion of the light chain constant region, or at least a portion of the heavy chain constant region and the light chain constant region, is or comprises amino acid sequence of a human, a rabbit, a mouse or a rat origin or comprises constant region amino acid sequence derived from a human, a rabbit, a mouse or a rat;
at least a portion of the heavy chain constant region, at least a portion of the light chain constant region, or at least a portion of the heavy chain constant region and the light chain constant region, is or comprises a synthetic amino acid sequence;
the recombinant Ab, the Ag binding fragment thereof, or monomeric or dimeric ABP, or the heavy chain constant region, or the light chain constant region, or the heavy chain constant region and the light chain constant region, further comprises or is bound to a heterologous protein, peptide, or a compound or a composition, and optionally the heterologous protein or peptide, or the compound or a composition, comprises a detectable protein, a detectable agent or a binding moiety, or, the heterologous protein or peptide comprises a carrier protein, or, the heterologous protein, peptide or the compound or composition, is covalently conjugated to the recombinant antibody (Ab), or Ag binding fragment thereof, or monomeric or dimeric ABP, and optionally the detectable agent or binding moiety comprises a biotin, a fluorescent or chemiluminescent label, a fluorophore, perylene, fluorenyl, coumarin, 7-methoxycoumarin (Mca), 4-(dimethylaminoazo)benzene-4-carboxylic acid (dabcyl), Tamra, boron-dipyrromethene (BODIPY), or derivatives thereof, a dye, a radioisotope, a quantum dot or photoluminescent aqueous nanocrystal, a hapten, or an antibody binding epitope or domain, and optionally the dye is or comprises rhodamine, [2-(4-nitro-2,1,3-benzoxadiazol-7-yl)aminoethyl]trimethylammonium (NBD), nile red or nile blue, or is a fluorescent dye comprising sulfoindocyanine, and optionally the fluorophore is or comprises a dansyl, a fluorescein, a carboxyfluorescein (FAM) or a 6-FAM moiety, and optionally the dye is or comprises a cyanine dye, a Cy3 or a Cy5, and optionally the hapten is or comprises a biotin, a theophylline, a digoxigenin, a carborane, a fluorescein or a bromodeoxyuridine moiety; and/or
the Ab, or Ag binding fragment thereof, or monomeric or dimeric ABP is a recombinant Ab, or Ag binding fragment thereof, or monomeric or dimeric ABP, or comprises a peptide or polypeptide made by a recombinant technique.

In alternative embodiments, provided are chimeric or recombinant nucleic acids comprising: a nucleic acid sequence encoding an antibody (Ab), or Ag binding fragment thereof, or monomeric or dimeric ABP as provided herein.

In alternative embodiments of chimeric or recombinant nucleic acids as provided herein:
the chimeric or recombinant nucleic acid further comprises and is operatively linked to a transcriptional regulatory element, and optionally the transcriptional regulatory element comprises a promoter, and optionally the promoter is an inducible promoter or a constitutive promoter;
the chimeric or recombinant nucleic acid further comprises sequence encoding an amino terminal signal peptide, and optionally the amino terminal signal peptide comprises the amino acid sequence:

(SEQ ID NO: 9)
METGLRWLLLVAVLKGVQC;
(SEQ ID NO: 10)
MDTRAPTQLLGLLLLWLPGATF;
(SEQ ID NO: 11)
METGLRWLLLVAVLKGVQC;
(SEQ ID NO: 12)
MDTRAPTQLLGLLLLWLPGAAF;
(SEQ ID NO: 13)
METGLRWLLLVAVLKGVQC;
or
(SEQ ID NO: 14)
MDTRAPTQLLGLLLLWLPGATF;

and/or
the amino terminal signal peptide SEQ ID NO:9 is amino terminal to VH SEQ ID NO:1; the amino terminal signal peptide SEQ ID NO:10 is amino terminal to VL SEQ ID NO:2; the amino terminal signal peptide SEQ ID NO:11 is amino terminal to VH SEQ ID NO:3; the amino terminal signal peptide to VL SEQ ID NO:12 is amino terminal to SEQ ID NO:4; the amino terminal signal peptide to VH SEQ ID NO:13 is amino terminal to SEQ ID NO:5, and the amino terminal signal peptide to VL SEQ ID NO:14 is amino terminal to SEQ ID NO:6.

In alternative embodiments, provided are expression cassettes, vectors, recombinant viruses, artificial chromosomes, cosmids or plasmids comprising a chimeric or a recombinant nucleic acid as provided herein.

In alternative embodiments, provided are cells comprising a chimeric or recombinant antibody or dimeric antigen binding protein as provided herein, or a chimeric or recombinant nucleic acid as provided herein, or an expression cassette, vector, recombinant virus, artificial chromosome, cosmid or plasmid as provided herein.

In alternative embodiments, of cells as provided herein:
the cell is a bacterial, fungal, mammalian, yeast, insect or plant cell, or a lymphocyte, or a T cell;
the cell expresses or secretes the chimeric or recombinant antibody or dimeric antigen binding protein extracellularly or into the extracellular milieu, or at least the antigen binding moiety of the chimeric or recombinant antibody or dimeric antigen binding protein cell is expressed or displayed extracellularly or into the extracellular milieu; and/or
the mammalian cell is a human cell, or a human lymphocyte, or a human T cell.

In alternative embodiments, provided are methods for detecting the presence of a human B-cell maturation antigen (BCMA, or BCM) protein or polypeptide in or on a cell, a tissue, an organ or a portion of any of the foregoing, comprising:
(a) contacting the cell, tissue or organ or portion of any of the foregoing with at least one Ab, or Ag binding fragment thereof, or monomeric or dimeric ABP as provided herein, and
(b) detecting the specific binding of the at least one Ab, or Ag binding fragment thereof, or monomeric or dimeric ABP with a BCMA polypeptide, in or on the cell, tissue or organ or portion of any of the foregoing,
thereby detecting the presence of the human BCMA protein in or on the cell, tissue, organ or portion of any of the foregoing.

In alternative embodiments of methods as provided herein:
the at least one Ab, or Ag binding fragment thereof, or monomeric or dimeric ABP comprises a variable heavy chain (VH) having an amino acid sequence comprising SEQ ID NO:1 and a variable light chain (VL) having an amino acid sequence comprising SEQ ID NO:2;
the at least one Ab, or Ag binding fragment thereof, or monomeric or dimeric ABP comprises a variable heavy chain (VH) having an amino acid sequence comprising SEQ ID NO:3 and a variable light chain (VL) having an amino acid sequence comprising SEQ ID NO:4;
the at least one Ab, or Ag binding fragment thereof, or monomeric or dimeric ABP comprises a variable heavy chain (VH) having an amino acid sequence comprising SEQ ID NO:5 and a variable light chain (VL) having an amino acid sequence comprising SEQ ID NO:6;
the method comprises contacting the cell, tissue or organ or portion of any of the foregoing with two Abs, or Ag binding fragments thereof, or monomeric or dimeric ABPs, or a mixture of two Abs, or Ag binding fragments thereof, or monomeric or dimeric ABPs;
one of the two Abs, or Ag binding fragments thereof, or monomeric or dimeric ABPs comprises a variable heavy chain (VH) having an amino acid sequence comprising SEQ ID NO:1 and a variable light chain (VL) having an amino acid sequence comprising SEQ ID NO:2; and the second of the two Abs, or Ag binding fragments thereof, or monomeric or dimeric ABPs comprises a variable heavy chain (VH) having an amino acid sequence comprising SEQ ID NO:3 and a variable light chain (VL) having an amino acid sequence comprising SEQ ID NO:4;
one of the two Abs, or Ag binding fragments thereof, or monomeric or dimeric ABPs comprises a variable heavy chain (VH) having an amino acid sequence comprising SEQ ID NO:3 and a variable light chain (VL) having an amino acid sequence comprising SEQ ID NO:4; and the second of the two Abs, or Ag binding fragments thereof, or monomeric or dimeric ABPs comprises a variable heavy chain (VH) having an amino acid sequence comprising SEQ ID NO:5 and a variable light chain (VL) having an amino acid sequence comprising SEQ ID NO:6;
one of the two Abs, or Ag binding fragments thereof, or monomeric or dimeric ABPs comprises a variable heavy chain (VH) having an amino acid sequence comprising SEQ ID NO:1 and a variable light chain (VL) having an amino acid sequence comprising SEQ ID NO:2; and the second of the two Abs, or Ag binding fragments thereof, or monomeric or dimeric ABPs comprises a variable heavy chain (VH) having an amino acid sequence comprising SEQ ID NO:5 and a variable light chain (VL) having an amino acid sequence comprising SEQ ID NO:6;
the contacting comprises use of an immunohistochemistry (IHC) assay;
the method further comprises contacting the Ab, or Ag binding fragment thereof, or monomeric or dimeric ABP, specifically bound to a BCMA polypeptide, with a detectable agent to indicate or signal the specific binding of the Ab, or Ag binding fragment thereof, or monomeric or dimeric ABP, to the human BCMA protein;
the detectable agent specifically binds to the Ab, or Ag binding fragment thereof, or monomeric or dimeric ABP;
the cell, tissue, organ or a portion of any of the foregoing is or comprises: a lymphocyte, and optionally the lymphocyte comprises or is a B-cell or B lymphocyte, and optionally the B-cell or B lymphocyte comprises or is an early B cell, a pro-B cell, a pre-B lymphocyte, a mature B-lymphocyte, and optionally the lymphocyte comprises or is a T-cell or T lymphocyte, and optionally the T-cell or T lymphocyte comprises or is a parafollicular T lymphocyte or a subpopulation of parafollicular T lymphocytes;
the cell, tissue, organ or a portion of any of the foregoing is or comprises: a cancer or malignant cell, and optionally the cancer or malignant cell comprises or is a breast myoepithelial cell, a leukemia cell, a carcinoma cell, a carcinoid tumor cell, a mamma carcinoma cell, a colon carcinoma cell, a malignant melanoma cell, a multiple myeloma cell, a plasmacytoma cell or a lymphoma cell, and optionally the cell, tissue, organ or a portion of any of the foregoing is or comprises: a follicular center cell, or a cell in a tonsil, an organ, a lymph node germinal center, a bone marrow stem cell, a myelopoietic cell, a liver bile canalicular cell, a renal glomerular cell, a proximal tubular cell, a stromal cell around or associated with an infiltrating tumor cell, a kidney cell, an epithelial cell, a cerebellum cell, a prostate cell, a kidney cell, a pancreas cell, or a bone marrow cell, and optionally the epithelial cell is or is derived from a brain, lung, intestine, kidney, breast or placental epithelial cell, and optionally the organ is a liver, prostate or lung, and optionally the leukemia cell is an acute lymphoblastic leukemia (ALL) cell;
the carcinoma cell is a basal cell carcinoma (BCC) cell; and/or
the lymphoma is a B cell lymphoma, and optionally the B cell lymphoma is a Hodgkin's lymphoma or a non-Hodgkin's lymphoma, and optionally the non-Hodgkin's lymphoma is follicular lymphoma, a diffuse large B cell lymphoma, a marginal zone B cell lymphoma, a small lymphocytic lymphoma (SLL), a chronic lymphocytic leukemia, (CLL), a Burkitt lymphoma or a mantle cell lymphoma (MCL).

In alternative embodiments, provided are methods for detecting or diagnosing a cancer,

wherein the method comprises detecting expression or presence of a human BCMA protein or peptide in or on a cell, tissue or organ sample using a method as provided herein,

wherein the detecting of the specific binding of the Ab, or Ag binding fragment thereof, or monomeric or dimeric ABP with a BCMA polypeptide in or on the cell, tissue or organ or portion of any of the foregoing, detects or diagnoses, or assists in the detection or diagnosis of, the cancer.

In alternative embodiments of methods for detecting or diagnosing a cancer as provided herein:
the cancer is: leukemic cell cancer of pre-B phenotype, a lymphoma, a plasmocytoma, chronic myelogenous leukemia in blast crisis, diffuse large B-cell lymphoma, hairy cell leukemia, a myeloma, precursor T lymphoblastic leukemia or lymphoma, a non-hematolymphoid sarcoma, or a carcinoma;
the lymphoma is a B cell lymphoma, a precursor B lymphoblastic leukemia or lymphoma, a follicular lymphoma, an acute lymphocytic leukemia (ALL), an angioimmunoblastic T cell lymphoma, a Burkitt's lymphoma, a diffuse large B-cell lymphoma, a mantle cell lymphoma or an angioimmunoblastic T-cell lymphoma;
the B cell lymphoma is a Hodgkin's lymphoma or a non-Hodgkin's lymphoma, and optionally the non-Hodgkin's lymphoma is follicular lymphoma, a diffuse large B cell lymphoma, a marginal zone B cell lymphoma, a small lymphocytic lymphoma (SLL) or chronic lymphocytic leukemia, (CLL), or a mantle cell lymphoma (MCL);
the carcinoma is a renal cell carcinoma or a metaplastic breast carcinoma;
the cell, tissue or organ sample is from an individual in need thereof;
the detection comprises conducting an immunohistochemistry (IHC) assay;
the at least two Abs, or Ag binding fragments thereof, or monomeric or dimeric ABPs, are used to contact the cell, tissue or organ sample; and/or
one of the two Abs, or Ag binding fragments thereof, or monomeric or dimeric ABPs comprises a variable heavy chain (VH) having an amino acid sequence comprising SEQ ID NO:3 and a variable light chain (VL) having an amino acid sequence comprising SEQ ID NO:4; and the second of the two Abs, or Ag binding fragments thereof, or monomeric or dimeric ABPs comprises a variable heavy chain (VH) having an amino acid sequence comprising SEQ ID NO:5 and a variable light chain (VL) having an amino acid sequence comprising SEQ ID NO:6.

In alternative embodiments, provided are methods for treating, ameliorating or preventing a cancer comprising first detecting or diagnosing the cancer using a method as provided herein, followed by treatment of the individual in need thereof for the treatment, amelioration or prevention of the cancer.

In alternative embodiments of methods for treating, ameliorating or preventing a cancer as provided herein:
the cancer is: leukemic cell cancer of pre-B phenotype, a lymphoma, chronic myelogenous leukemia in blast crisis, diffuse large B-cell lymphoma, hairy cell leukemia, a myeloma, a precursor B lymphoblastic leukemia or lymphoma, a follicular lymphoma, precursor T lymphoblastic leukemia or lymphoma, a non-hematolymphoid sarcoma, or a carcinoma, and optionally the lymphoma is a B cell lymphoma, acute lymphocytic leukemia (ALL), angioimmunoblastic T cell lymphoma, a Burkitt's lymphoma, diffuse large B-cell lymphoma, mantle cell lymphoma, a plasmocytoma, angioimmunoblastic T-cell lymphoma, and optionally the B cell lymphoma is a Hodgkin's lymphoma or a non-Hodgkin'lymphoma, and optionally the non-Hodgkin's lymphoma is follicular lymphoma, a diffuse large B cell lymphoma, a marginal zone B cell lymphoma, a small lymphocytic lymphoma (SLL) or chronic lymphocytic leukemia, (CLL), or a mantle cell lymphoma (MCL), and optionally the carcinoma is a renal cell carcinoma or a metaplastic breast carcinoma.

In alternative embodiments, provided are uses of at least one recombinant antibody (Ab), or antigen (Ag) binding fragment thereof, or monomeric or dimeric antigen binding protein (ABP) as provided herein, for detecting or diagnosing a cancer, or treating, ameliorating or preventing a cancer.

In alternative embodiments of uses of at least one recombinant antibody (Ab), or antigen (Ag) binding fragment thereof, or monomeric or dimeric antigen binding protein (ABP) as provided herein:
the cancer is: leukemic cell cancer of pre-B phenotype, a lymphoma, chronic myelogenous leukemia in blast crisis, diffuse large B-cell lymphoma, hairy cell leukemia, a myeloma, a precursor B lymphoblastic leukemia or lymphoma, a follicular lymphoma, precursor T lymphoblastic leukemia or lymphoma, a non-hematolymphoid sarcoma, or a carcinoma, and optionally the lymphoma is a B cell lymphoma, a plasmocytoma, acute lymphocytic leukemia (ALL), angioimmunoblastic T cell lymphoma, a Burkitt's lymphoma, diffuse large B-cell lymphoma, mantle cell lymphoma, angioimmunoblastic T-cell lymphoma, and optionally the B cell lymphoma is a Hodgkin's lymphoma or a non-Hodgkin's lymphoma, and optionally the non-Hodgkin's lymphoma is follicular lymphoma, a diffuse large B cell lymphoma, a marginal zone B cell lymphoma, a small lymphocytic lymphoma (SLL) or chronic lymphocytic leukemia, (CLL), or a mantle cell lymphoma (MCL), and optionally the carcinoma is a renal cell carcinoma or a metaplastic breast carcinoma; and/or
the detection comprises conducting an immunohistochemistry (IHC) assay,
recombinant antibodies (Abs), or antigen (Ag) binding fragments thereof, or monomeric or dimeric antigen binding proteins (ABPs) (as described herein) for use in detecting or diagnosing a cancer, or treating, ameliorating or preventing a cancer.

In alternative embodiments of recombinant antibodies (Abs), or antigen (Ag) binding fragments thereof, or monomeric or dimeric antigen binding proteins (ABPs) as provided herein:
the cancer is: leukemic cell cancer of pre-B phenotype, a lymphoma, chronic myelogenous leukemia in blast crisis, diffuse large B-cell lymphoma, hairy cell leukemia, a myeloma, a precursor B lymphoblastic leukemia or lymphoma, a follicular lymphoma, precursor T lymphoblastic leukemia or lymphoma, a non-hematolymphoid sarcoma, or a carcinoma, and optionally the lymphoma is a B cell lymphoma, acute lymphocytic leukemia (ALL), angioimmunoblastic T cell lymphoma, Burkitt lymphoma, a Burkitt's lymphoma, diffuse large B-cell lymphoma, mantle cell lymphoma, a plasmocytoma, angioimmunoblastic T-cell lymphoma, and optionally the B cell lymphoma is a Hodgkin's lymphoma or a non-Hodgkin's lymphoma, and optionally the non-Hodgkin's lymphoma is follicular lymphoma, a diffuse large B cell lymphoma, a marginal zone B cell lymphoma, a small lymphocytic lymphoma (SLL) or chronic lymphocytic leukemia, (CLL), or a mantle cell lymphoma (MCL), and optionally the carcinoma is a renal cell carcinoma or a metaplastic breast carcinoma; and/or
the detecting or diagnosing comprises conducting an immunohistochemistry (IHC) assay.

In alternative embodiments, provided are products of manufacture, or kits, comprising: a chimeric or recombinant antibody as provided herein; a chimeric or a recombinant nucleic acid as provided herein; or an expression cassette, vector, recombinant virus, artificial chromosome, cosmid or plasmid as provided herein. The product of manufacture, or kit, can comprise components needed for an immunohistochemistry (IHC) assay, and/or comprises instructions for practicing a method as provided herein. In alternative embodiments, the products of manufacture, or kits, comprise mixtures or cocktails of antibodies (Abs) as provided herein, for example, a mixture or cocktail comprising an anti-human BCMA Ab produced by clone 9H5 (comprising a variable heavy chain (VH) having an amino acid sequence comprising SEQ ID NO:3 and a variable light chain (VL) having an amino acid sequence comprising SEQ ID NO:4) an anti-human BCMA Ab produced by clone 10A2 (comprising a variable heavy chain (VH) having an amino acid sequence comprising SEQ ID NO:5 and a variable light chain (VL) having an amino acid sequence comprising SEQ ID NO:6).

The details of one or more exemplary embodiments of the invention are set forth in the accompanying drawings and the description below. Other features, objects, and advantages of the invention will be apparent from the description and drawings, and from the claims.

All publications, patents, patent applications cited herein are hereby expressly incorporated by reference in their entireties for all purposes.

### DESCRIPTION OF DRAWINGS

The patent or application file contains at least one drawing executed in color. Copies of this patent or patent application publication with color drawing(s) will be provided by the Office upon request and payment of the necessary fee.

The drawings set forth herein are illustrative of exemplary embodiments provided herein and are not meant to limit the scope of the invention as encompassed by the claims.

FIG. 1A (FIG. 1a) illustrates an image of a supersensitive IHC of B cell clone 9H5 on normal colon. Plasma cells in lamina propria of the colon epithelia show membrane and cytoplasmatic staining.

FIG. 1B (FIG. 1b) illustrates an image of a supersensitive IHC of B cell clone 10A2 on normal colon. Plasma cells in lamina propria of the colon epithelia show membrane and cytoplasmatic staining.

FIG. 1C (FIG. 1c) illustrates an image of a supersensitive IHC of B cell clone 5C1 on normal colon. Plasma cells in lamina propria of the colon epithelia show membrane and cytoplasmatic staining.

FIG. 2A (FIG. 2a) illustrates an image of a supersensitive IHC of B cell clone 9H5 on normal tonsil. Germinal center B cells, plasmablasts and plasma cells below and within the tonsillar epithelium (bottom left corner) show membrane and cytoplasmatic staining of different intensity.

FIG. 2B (FIG. 2b) illustrates an image of a supersensitive IHC of B cell clone 10A2 on normal tonsil. Germinal center B cells, plasmablasts and plasma cells below and within the tonsillar epithelium (bottom left corner) show membrane and cytoplasmatic staining of different intensity.

FIG. 2C (FIG. 2c) illustrates an image of a supersensitive IHC of B cell clone 5C1 on normal tonsil. Germinal center B cells, plasmablasts show membrane and cytoplasmatic staining of different intensity.

FIG. 3A (FIG. 3a) illustrates an image of a supersensitive ICH of B cell clone 9H5 on BCMA positive cell line U-2932. Strong to medium membrane staining of virtually all cells, confirming specificity of the BCMA clone 9H5.

FIG. 3B (FIG. 3b) illustrates an image of a supersensitive ICH of B cell clone 10A2 on BCMA positive cell line U-2932. Strong to medium membrane staining of virtually all cells, confirming the specificity of the BCMA clone 10A2.

FIG. 4A (FIG. 4a) illustrates an image of a standard IHC (Envision FLEX™) of 5 clone 9H5 alone on reactive lymph node. Moderate cytoplasmatic and membrane staining of plasma cells.

FIG. 4B (FIG. 4b) illustrates an image of a standard IHC (Envision FLEX™) of clone 10A2 alone on reactive lymph node. Strong to moderate cytoplasmatic and membrane staining of plasma cells.

FIG. 4C (FIG. 4c) illustrates an image of a standard IHC (Envision FLEX™) of mix (0.25 antibody concentration) of clone 9H5 and 10A2 on reactive lymph node. Strong to moderate, distinct cytoplasmatic and membrane staining of plasma cells.

FIG. 4D (FIG. 4d) illustrates an image of a supersensitive IHC of clone 5C1 on reactive lymph node. Strong cytoplasmatic and membrane staining of plasma cells.

FIG. 5A (FIG. 5a) illustrates an image of a standard IHC (Envision FLEX™) of clone 9H5 alone on plasmacytoma. Strong to moderate, primarily membrane and few Golgi staining of plasmacytoma tumor cells.

FIG. 5B (FIG. 5b) illustrates an image of a standard IHC (Envision FLEX™) of clone 10A2 alone on plasmacytoma. Strong primarily membrane and some Golgi staining of plasmacytoma tumor cells.

FIG. 5C (FIG. 5c) illustrates an image of a standard IHC (Envision FLEX™) of mix (0.25 antibody concentration) of clone 9H5 and 10A2 on plasmacytoma. Strong and distinct, primarily membrane and some Golgi staining of plasmacytoma tumor cells.

FIG. 5D (FIG. 5d) illustrates an image of a supersensitive IHC of clone 5C1 on plasmacytoma. Strong to moderate, membrane and many strong Golgi staining of plasmacytoma tumor cells.

FIG. 6A (FIG. 6a) illustrates an image of a standard IHC (Envision FLEX™) of clone 9H5 alone on multiple myeloma. Strong to moderate, primarily membrane staining of myeloma tumor cells.

FIG. 6B (FIG. 6b) illustrates an image of a standard IHC (Envision FLEX™) of clone 10A2 alone on multiple myeloma. Strong primarily membrane staining of myeloma tumor cells.

FIG. 6C (FIG. 6c) illustrates an image of a standard IHC (Envision FLEX™) of mix (0.25 antibody concentration) of clone 9H5 and 10A2 on multiple myeloma. Strong and distinct, primarily membrane staining of myeloma tumor cells.

Further tests were made to determine the effect of mixing the 2 extracellular antibody clones 9H5 and 10A2. An example of an optimal protocol of the mix of the two antibodies in standard IHC (EnVision FLEX™) and of the intracellular clone 5C1 alone, is illustrated in FIGS. 7-11 on multiple myeloma, plasmacytoma, Burkitt lymphoma and positive and negative cell line.

FIG. 7A (FIG. 7a) illustrates an image of a standard IHC optimal protocol of mix of clone 9H5 and 10A2 on multiple myeloma. Distinct membrane and Golgi staining of myeloma tumour cells.

FIG. 7B (FIG. 7b) illustrates an image of a standard IHC optimal protocol of clone 5C1 on multiple myeloma. Distinct membrane and Golgi staining of myeloma tumour cells.

FIG. 8A (FIG. 8a) illustrates an image of a standard IHC optimal protocol of mix of clone 9H5 and 10A2 on plasmacytoma. Distinct membrane and strong Golgi staining of plasmacytoma tumour cells.

FIG. 8B (FIG. 8b) illustrates an image of a standard IHC optimal protocol of clone 5C1 on plasmacytoma. Moderate cytoplasmatic, a few membrane and strong Golgi staining of plasmacytoma tumour cells.

FIG. 9A (FIG. 9a) illustrates an image of a standard IHC optimal protocol of mix of clone 9H5 and 10A2 on Burkitt lymphoma. Distinct membrane and Golgi staining of tumour cells.

FIG. 9B (FIG. 9b) illustrates an image of a standard IHC optimal protocol of clone 5C1 on Burkitt lymphoma. Distinct membrane and Golgi staining of tumour cells.

FIG. 10A (FIG. 10a) illustrates an image of a standard IHC optimal protocol of mix of clone 9H5 and 10A2 on BCMA positive high expressing cell line NCI-H929.

FIG. 10B (FIG. 10v) illustrates an image of a standard IHC optimal protocol of clone 5C1 on BCMA positive high expressing cell line U-2932.

FIG. 11A (FIG. 11a) illustrates an image of a standard IHC optimal protocol of mix of clone 9H5 and 10A2 on BCMA negative cell line MCF-7.

FIG. 11B (FIG. 11b) illustrates an image of a standard IHC optimal protocol of clone 5C1 on BCMA negative cell line MCF-7.

Like reference symbols in the various drawings indicate like elements.

### DETAILED DESCRIPTION

In alternative embodiments, provided are isolated, recombinant or purified antibodies (Abs), or antigen (Ag) binding fragments thereof, or monomeric or dimeric antigen binding proteins (ABPs), capable of specifically binding a human B-cell maturation antigen (BCMA, or BCM) polypeptide (also called tumor necrosis factor receptor superfamily member 17 (TNFRSF17)), including nucleic acids that encode them, cells that express them, and methods for making an using them, including methods for practicing immunohistochemistry (IHC) methodologies, and diagnosing and detecting cancer.

In alternative embodiments, provided are products of manufacture such as kits comprising or having contained therein isolated, recombinant or purified antibodies (Abs), or antigen (Ag) binding fragments thereof, or monomeric or dimeric antigen binding proteins (ABPs), capable of specifically binding BCMA, for use in, for example, IHC assays. In alternative embodiments, the products of manufacture, or kits, comprise mixtures or cocktails of antibodies (Abs) as provided herein, for example, a mixture or cocktail comprising an anti-human BCMA Ab produced by clone 9H5 (comprising a variable heavy chain (VH) having an amino acid sequence comprising SEQ ID NO:3 and a variable light chain (VL) having an amino acid sequence comprising SEQ ID NO:4) and an anti-human BCMA Ab produced by clone 10A2 (comprising a variable heavy chain (VH) having an amino acid sequence comprising SEQ ID NO:5 and a variable light chain (VL) having an amino acid sequence comprising SEQ ID NO:6). In alternative embodiments, the products of manufacture, or kits, comprise mixtures or cocktails of antibodies (Abs) as provided herein comprising Ab heavy and light claims having CDR sequences comprising the specific CDR sequences of Ab sequences and Ab producing clones as provided herein.

A BCMA antigen was designed by using the entire extracellular domain of BCMA in fusion to rabbit Fc domain and expressing the recombinant antigen in HEK cells. The antigen was used for immunizations of rabbits and bleeds were later taken to confirm serum reactivity against BCMA in ELISA and IHC. The rabbits showing best immune response against BCMA were chosen for B-cell selection using blood samples from the rabbits. Rabbit anti-BCMA antibodies were tested for specificity on different clinical tissues showing usefulness for in vitro diagnostics by immunohistochemistry.

### Expression of Recombinant Chimeric Antibodies

In alternative embodiments, chimeric or recombinant Abs as provided herein, including the exemplary chimeric or recombinant anti-human BCMA Ab, with signal peptide, or without the signal peptide, can be expressed as a recombinant Ab using a plasmid (or any expression vehicle) encoding the respective heavy and light chains, or the heavy chain and the light chain can be encoded in separate expression vehicles.

In some embodiments, the heavy and light chains can be (cis- or trans-) expressed from a pTT5™ vector(s) (National Research Council Canada, NRC-CNRC, Canada) in HEK293-6E cells. In alternative embodiment, the vector or vectors expressing the heavy and/or light chains are episomal or are chromosomally integrated, for example, in a stable cell line capable of synthesizing, optionally inducibly synthesizing, the heavy and/or light chains.

In alternative embodiments, provided are nucleic acids encoding chimeric or recombinant Abs as provided herein. Nucleic acids as provided herein can be made, isolated and/or manipulated by, for example, cloning and expression of cDNA libraries, amplification of message or genomic DNA by PCR, and the like. Nucleic acids used to practice embodiments as provided herein, whether RNA, cDNA, genomic DNA, vectors, viruses or hybrids thereof, may be isolated from a variety of sources, genetically engineered, amplified, and/or expressed/ generated recombinantly. Recombinant polypeptides generated from these nucleic acids can be individually isolated or cloned and tested for a desired activity. Any recombinant expression system can be used, including bacterial, fungal, mammalian, yeast, insect or plant cell expression systems.

Alternatively, these nucleic acids can be synthesized in vitro by well-known chemical synthesis techniques, as described in, for example, Adams (1983) J. Am. Chem. Soc. 105:661; Belousov (1997) Nucleic Acids Res. 25:3440-3444; Frenkel (1995) Free Radic. Biol. Med. 19:373-380; Blommers (1994) Biochemistry 33:7886-7896; Narang (1979) Meth. Enzymol. 68:90; Brown (1979) Meth. Enzymol. 68:109; Beaucage (1981) Tetra. Lett. 22:1859; U.S. Pat. No. 4,458,066.

Techniques for the manipulation of nucleic acids, such as, for example, subcloning, labeling probes (for example, random-primer labeling using Klenow polymerase, nick translation, amplification), sequencing, hybridization and the like are well described in the scientific and patent literature, see, for example, Sambrook, ed., MOLECULAR CLONING: A LABORATORY MANUAL (2ND ED.), Vols. 1-3, Cold Spring Harbor Laboratory, (1989); CURRENT PROTOCOLS IN MOLECULAR BIOLOGY, Ausubel, ed. John Wiley & Sons, Inc., New York (1997); LABORATORY TECHNIQUES IN BIOCHEMISTRY AND MOLECULAR BIOLOGY: HYBRIDIZATION WITH NUCLEIC ACID PROBES, Part I. Theory and Nucleic Acid Preparation, Tijssen, ed. Elsevier, N.Y. (1993).

Another useful means of obtaining and manipulating nucleic acids used to practice embodiments as provided herein comprises screening and re-cloning inserts isolated or amplified from, for example, genomic clones or cDNA clones. Sources of nucleic acids include recombinant nucleic acid sequences, genomic or cDNA libraries contained and/or expressed in, for example, mammalian artificial chromosomes (MACs), see, for example, U.S. Pat. Nos. 5,721,118; 6,025,155; human artificial chromosomes, see, for example, Rosenfeld (1997) Nat. Genet. 15:333-335; yeast artificial chromosomes (YAC); bacterial artificial chromosomes (BAC); P1 artificial chromosomes, see, for example, Woon (1998) Genomics 50:306-316; P1-derived vectors (PACs), see, for example, Kern (1997) Biotechniques 23:120-124; cosmids, recombinant viruses, phages, phagemids or plasmids.

In alternative embodiments, nucleic acids as provided herein are operably linked to transcriptional regulatory elements, including promoters, with can be constitutive or inducible transcriptional regulatory elements.

In alternative aspects, provided are “expression cassettes” comprising a nucleotide sequence as provided herein, for example encoding a chimeric or recombinant antibody as provided herein. Expression cassettes can include at least a transcriptional regulatory element, for example, a promoter, operably linked with an antibody coding sequence, and optionally can also include transcription termination signals. Additional factors necessary or helpful in effecting expression may also be used, for example, enhancers.

In alternative aspects, expression cassettes used to practice embodiments as provided herein include plasmids, expression vectors, recombinant viruses, any form of recombinant “naked DNA” vector, and the like. In alternative aspects, a “vector” used to practice embodiments as provided herein can comprise a nucleic acid that can infect, transfect, transiently or permanently transduce a cell. In alternative aspects, a vector used to practice embodiments as provided herein can be a naked nucleic acid, or a nucleic acid complexed with protein or lipid. In alternative aspects, vectors used to practice embodiments as provided herein can comprise viral or bacterial nucleic acids and/or proteins, and/or membranes (for example, a cell membrane, a viral lipid envelope, etc.). In alternative aspects, vectors used to practice embodiments as provided herein can include, but are not limited to replicons (for example, RNA replicons, bacteriophages) to which fragments of DNA may be attached and become replicated. Vectors thus include, but are not limited to RNA, autonomous self-replicating circular or linear DNA or RNA (for example, plasmids, viruses, and the like, see, for example, U.S. Pat. No. 5,217,879), and can include both the expression and non-expression plasmids. In alternative aspects, the vector used to practice embodiments as provided herein can be stably replicated by the cells during mitosis as an autonomous structure, or can be incorporated within the host's genome.

In alternative aspects, “promoters” used to practice embodiments as provided herein include all sequences capable of driving transcription of a coding sequence in a cell, for example, a bacterial, yeast, fungal, plant, insect (for example, baculovirus) or mammalian cell. Thus, promoters used in the constructs include cis-acting transcriptional control elements and regulatory sequences that are involved in regulating or modulating the timing and/or rate of transcription of a gene. For example, a promoter used to practice embodiments as provided herein can be a cis-acting transcriptional control element, including an enhancer, a promoter, a transcription terminator, an origin of replication, a chromosomal integration sequence, 5′ and 3′ untranslated regions, or an intronic sequence, which are involved in transcriptional regulation. These cis-acting sequences can interact with proteins or other biomolecules to carry out (turn on/off, regulate, modulate, etc.) transcription.

“Constitutive” promoters used to practice embodiments as provided herein can be those that drive expression continuously under most environmental conditions and states of development or cell differentiation. “Inducible” or “regulatable” promoters used to practice embodiments as provided herein can direct expression of a nucleic acid as provided herein under the influence of environmental conditions or developmental conditions. Examples of environmental conditions that may affect transcription by inducible promoters used to practice embodiments as provided herein include the presence of an inducing factor administered to a cell.

In alternative embodiments, peptides and polypeptides used to practice embodiments as provided herein can comprise any “mimetic” and/or “peptidomimetic” form. In alternative embodiments, peptides and polypeptides used to practice embodiments as provided herein can comprise synthetic chemical compounds which have substantially the same structural and/or functional characteristics of the natural polypeptide, for example, a chimeric or recombinant antibody as provided herein. The mimetic used to practice embodiments as provided herein can be either entirely composed of synthetic, non-natural analogues of amino acids, or, is a chimeric molecule of partly natural peptide amino acids and partly non-natural analogs of amino acids. The mimetic can also incorporate any amount of natural amino acid conservative substitutions as long as such substitutions also do not substantially alter the mimetic's structure and/or activity. Routine experimentation will determine whether a mimetic is effective for practicing the invention, for example, if a mimetic composition is effective in specifically binding BCMA protein. Methodologies detailed herein and others known to persons skilled in the art may be used to select or guide one to choose effective mimetic for practicing the compositions and/or methods of this invention.

Polypeptide mimetic compositions for practicing embodiments as provided herein can comprise any combination of non-natural structural components. In alternative aspects, mimetic compositions for practicing embodiments as provided herein can comprise one or all of the following three structural groups: a) residue linkage groups other than the natural amide bond (“peptide bond”) linkages; b) non-natural residues in place of naturally occurring amino acid residues; or c) residues which induce secondary structural mimicry, i.e., to induce or stabilize a secondary structure, for example, a beta turn, gamma turn, beta sheet, alpha helix conformation, and the like. For example, a polypeptide can be characterized as a mimetic when all or some of its residues are joined by chemical means other than natural peptide bonds.

### Immunohistochemistry

In alternative embodiments, immunohistochemistry (IHC) methodologies and/or reagents used to practice compositions, products of manufacture, kits or methods as provided herein can include or comprise or comprise use of any IHC protocol, IHC armamentarium, device and/or image or data analysis system, for practicing IHC or IHC reagents known in the art, for example, as described in U.S. Pat. No. 10,565,479 (describing methods for identifying blurred areas in digital images of stained tissue); U.S. Pat. No. 10,564,076 (describing systems for analytical (or IHC) sample preparation); U.S. Pat. No. 10,551,395 (describing an automated histological staining system); U.S. Pat. No. 10,551,378 (describing a tissue staining method); U.S. Pat. No. 10,504,224 (describing a digital tissue image analysis system for IHC); U.S. Pat. No. 10,501,777 (describing simultaneous, multiplexed detection and quantification of protein expression in IHC); U.S. Pat. No. 10,488,340 (describing method for extracting an image of a target fluorophore in a biological material); U.S. Pat. No. 10,453,195 (describing methods of detecting tissue areas of interest using digital pathology imaging); U.S. Pat. No. 10,438,381 (describing devices, systems and methods for generating a digital image of a tissue section); U.S. Pat. No. 10,416,176 (describing methods for processing specimens in an automated histological staining system); U.S. Pat. No. 10,393,633 (describing methods for processing and inhibiting the degradation of an IHC sample); U.S. Pat. No. 10,217,011 (describing handling of IHC slides); U.S. Pat. No. 10,209,165 (describing automated or semi-automated methods for assessing the quality of staining of a specimen containing cells); U.S. Pat. No. 10,126,216 (describing methods for fixing tissue samples for IHC); U.S. Pat. No. 9,423,322.

In alternative embodiments, chimeric or the recombinant antibodies, antigen binding fragments thereof, or monomeric or dimeric antigen binding proteins, in IHC protocols, or kits, as provided herein are substantially purified or isolated or are in the form of an unpurified or partially purified culture supernatant.

In alternative embodiments, methods as provided herein can use or comprise reagents for detecting or visualizing an antibody-antigen interaction using any products or methods know in the art, for example, and IHC protocol or reagents.

In alternative embodiments, methods as provided herein comprise use of chromogenic immunohistochemistry (CIH), wherein a primary antibody (for example, chimeric or a recombinant antibodies (Ab), or antigen binding fragments thereof, or monomeric or dimeric antigen binding proteins as provided herein) or secondary antibody (for example, where the secondary antibody binds to (the primary antibody) chimeric or a recombinant antibodies (Ab), or antigen binding fragments thereof, or monomeric or dimeric antigen binding proteins as provided herein after they have specifically bound to, paired with or associated with, a BCMA epitope or polypeptide) is conjugated to an enzyme, such as peroxidase (or immunoperoxidase), for example, a horseradish peroxidase (HRP), that can catalyze a color-producing reaction.

In alternative embodiments, methods as provided herein comprise use of immunofluorescence, where a primary or a secondary antibody is tagged to a fluorophore, such as fluorescein or fluorescein isothiocyanate (FITC), a triarylmethane dye such as rhodamine or rhodamine derivatives (for example, tetramethylrhodamine (TRITC), rhodamine 6G, rhodamine 123, rhodamine B, carboxytetramethylrhodamine (TAMRA), tetramethylrhodamine (TMR), sulforhodamine 101), aminomethylcoumarin acetate (AMCA), ALEXA™ or DYLIGHT™ fluors. 3,3′-Diaminobenzidine (DAB) also can be used.

In alternative embodiments, methods as provided herein comprise use of a direct method or one-step staining method where a primary antibody (for example, chimeric or a recombinant antibodies (Ab), or antigen binding fragments thereof, or monomeric or dimeric antigen binding proteins as provided herein) is labeled and reacts directly with an antigen, for example, in a tissue sections. While this technique utilizes only one antibody and therefore is simple and rapid, the sensitivity may be lower due to little signal amplification.

In alternative embodiments, methods as provided herein comprise use of an indirect method where an unlabeled primary antibody (first layer) binds to a target antigen (BCMA), for example, in a tissue or organ, and a labeled secondary antibody (second layer) then is reacted with the primary antibody. The secondary antibody can be against the isotype, for example, IgG, of the animal species in which the primary antibody is derived. This method can be more sensitive than direct detection strategies because of signal amplification due to the binding of several secondary antibodies to each primary antibody if the secondary antibody is conjugated to a detecting agent such as a fluorescent or enzyme reporter.

In alternative embodiments, further amplification is achieved if the secondary antibody is conjugated to several detecting molecules, for example, biotin molecules, which can recruit complexes of avidin-, streptavidin- or NEUTRAVIDIN™ protein-bound enzyme.

In alternative embodiments, the IHC is performed on tissue sections or tissue biopsies, for example, paraformaldehyde (PFA) fixed tissues or organs, or formalin-fixed paraffin-embedded tissues. In alternative embodiments, a tissue is sliced or used whole. Before sectioning, the tissue sample can be embedded in a medium, for example, paraffin wax or cryomedia. Tissue sections can be sliced on a variety of instruments, most commonly using a microtome, cryostat, or vibratome. Specimens can be sliced at a range of about 3 μm to 5 μm. The slices can be mounted on slides, dehydrated using alcohol washes of increasing concentrations (for example, 50%, 75%, 90%, 95%, 100%), and cleared using a detergent like xylene before being imaged under a microscope.

Depending on the method of fixation and tissue preservation, the sample may require additional steps to make the BCMA epitopes available for antibody binding, including deparaffinization and antigen retrieval. For formalin-fixed paraffin-embedded tissues, antigen-retrieval is often necessary, and can comprise pre-treating the sections with heat or proteases.

In alternative embodiments, the IHC is performed using an ENVISION DUOFLEX DOUBLESTAIN SYSTEM™ (EnVision DuoFLEX Doublestain System) (Agilent, San Jose, CA), which allows for staining of two or more markers on a single slide. In alternative embodiments, the IHC is performed using an EnVision FLEX HRP Magenta, High pH (Dako Omnis) system, and binding can be visualized by EnVision FLEX HRP™ Magenta Chromogen. In alternative embodiments, the IHC is performed using EnVision FLEX Mini Kit™, High pH, which is a high-sensitivity visualization system intended for use in IHC together with Dako AUTOSTAINER™instruments; this dual link system detects primary mouse and rabbit antibodies and the reaction is visualized by 3,3′-Diaminobenzidine (DAB) chromogen (DAB forms a water-insoluble brown precipitate when oxidized, for example, by a peroxidase)

### Products of Manufacture and Kits

Provided are products of manufacture and kits for practicing methods as provided herein, for example, comprising chimeric or recombinant anti-human B-cell maturation antigen (BCMA, or BCM) polypeptide (also called tumor necrosis factor receptor superfamily member 17 (TNFRSF17)) Abs as provided herein; and optionally the products of manufacture and kits can further comprise some or all reagents needed to perform an IHC, and optionally can comprise instructions for practicing methods as provided herein.

In alternative embodiments, the products of manufacture, or kits, comprise mixtures or cocktails of antibodies (Abs) as provided herein, for example, a mixture or cocktail comprising an anti-human BCMA Ab produced by clone 9H5 (comprising a variable heavy chain (VH) having an amino acid sequence comprising SEQ ID NO:3 and a variable light chain (VL) having an amino acid sequence comprising SEQ ID NO:4) an anti-human BCMA Ab produced by clone 10A2 (comprising a variable heavy chain (VH) having an amino acid sequence comprising SEQ ID NO:5 and a variable light chain (VL) having an amino acid sequence comprising SEQ ID NO:6).

In alternative embodiments, the products of manufacture, or kits, comprise mixtures or cocktails of antibodies (Abs) comprising an anti-human BCMA Ab produced by clone 9H5 and/or clone 10A2 and an anti-human BCMA Ab produced by clone 5C1 (comprising a variable heavy chain (VH) having an amino acid sequence comprising SEQ ID NO:5 and a variable light chain (VL) having an amino acid sequence comprising SEQ ID NO:6).

In alternative embodiments, the products of manufacture, or kits, comprise mixtures or cocktails of antibodies (Abs) comprising antibodies comprising heavy chain and/or light chain CDRs of antibodies produced by clones 9H5, 10A2 and/or 5C1, or comprising antibodies comprising heavy chain and/or light chain CDRs as provided herein, for example, comprising:
the three CDR1, CDR2 and CDR3 complementarity determining regions (CDRs) of SEQ ID NO:1, or CDR1 amino acid (aa) residues GFSLKTYV (residues 25-32 of SEQ ID NO:1), CDR2 aa residues IDTGDMT (residues 50 to 56 of SEQ ID NO:1), and CDR3 aa residues ASGDI (residues 95-99 of SEQ ID NO:1), and/or
the three CDR1, CDR2 and CDR3 complementarity determining regions (CDRs) of SEQ ID NO:2, or CDR1 amino acid (aa) residues QSVYSNW (residues 27-33 of SEQ ID NO:2), CDR2 aa residues SAS (residues 51 to 53 of SEQ ID NO:2), and CDR3 aa residues LGEFNCARADCRA (residues 90-102 of SEQ ID NO:2),
the three CDR1, CDR2 and CDR3 complementarity determining regions (CDRs) of SEQ ID NO:3, or CDR1 amino acid (aa) residues GFSLSAYA (residues 25-32 of SEQ ID NO:3), CDR2 aa residues INYDGIA (residues 50 to 56 of SEQ ID NO:3), and CDR3 aa residues ASDLLGVFNL (residues 93-102 of SEQ ID NO:3),
the three CDR1, CDR2 and CDR3 complementarity determining regions (CDRs) of SEQ ID NO:4, or CDR1 amino acid (aa) residues QSVYSNNR (residues 27-34 of SEQ ID NO:4), CDR2 aa residues YAS (residues 52 to 55 of SEQ ID NO:4), and CDR3 aa residues AGGYISYSDNA (residues 91-101 of SEQ ID NO:4),
the three CDR1, CDR2 and CDR3 complementarity determining regions (CDRs) of SEQ ID NO:5, or CDR1 amino acid (aa) residues GFSLTTYA (residues 25-32 of SEQ ID NO:5), CDR2 aa residues IWSSGTT (residues 50 to 56 of SEQ ID NO:5), and CDR3 aa residues ARYINYVTGDL (residues 93-103 of SEQ ID NO:5), and/or
the three CDR1, CDR2 and CDR3 complementarity determining regions (CDRs) of SEQ ID NO:6, or CDR1 amino acid (aa) residues QSVYNNVL (residues 27-34 of SEQ ID NO:6), CDR2 aa residues KAS (residues 52 to 54 of
SEQ ID NO:6), and CDR3 aa residues QGEFSCSSADCTA (residues 91-103 of SEQ ID NO:6).

Any of the above aspects and embodiments can be combined with any other aspect or embodiment as disclosed here in the Summary, Figures and/or Detailed Description sections.

As used in this specification and the claims, the singular forms “a,” “an” and “the” include plural referents unless the context clearly dictates otherwise.

Unless specifically stated or obvious from context, as used herein, the term “or” is understood to be inclusive and covers both “or” and “and”.

Unless specifically stated or obvious from context, as used herein, the term “about” is understood as within a range of normal tolerance in the art, for example within 2 standard deviations of the mean. About (use of the term “about”) can be understood as within 20%, 19%, 18%, 17%, 16%, 15%, 14%, 13%, 12% 11%, 10%, 9%, 8%, 7%, 6%, 5%, 4%, 3%, 2%, 1%, 0.5%, 0.1%, 0.05%, or 0.01% of the stated value. Unless otherwise clear from the context, all numerical values provided herein are modified by the term “about.”

Unless specifically stated or obvious from context, as used herein, the terms “substantially all”, “substantially most of”, “substantially all of” or “majority of” encompass at least about 75%, 80%, 85%, 90%, 91%, 92%, 93%, 94%, 95%, 96%, 97%, 98%, 99% or 99.5%, or more of a referenced amount of a composition.

The entirety of each patent, patent application, publication and document referenced herein hereby is incorporated by reference. Citation of the above patents, patent applications, publications and documents is not an admission that any of the foregoing is pertinent prior art, nor does it constitute any admission as to the contents or date of these publications or documents. Incorporation by reference of these documents, standing alone, should not be construed as an assertion or admission that any portion of the contents of any document is considered to be essential material for satisfying any national or regional statutory disclosure requirement for patent applications. Notwithstanding, the right is reserved for relying upon any of such documents, where appropriate, for providing material deemed essential to the claimed subject matter by an examining authority or court.

Modifications may be made to the foregoing without departing from the basic aspects of the invention. Although the invention has been described in substantial detail with reference to one or more specific embodiments, those of ordinary skill in the art will recognize that changes may be made to the embodiments specifically disclosed in this application, and yet these modifications and improvements are within the scope and spirit of the invention. The invention illustratively described herein suitably may be practiced in the absence of any element(s) not specifically disclosed herein. Thus, for example, in each instance herein any of the terms “comprising”, “consisting essentially of”, and “consisting of” may be replaced with either of the other two terms. Thus, the terms and expressions which have been employed are used as terms of description and not of limitation, equivalents of the features shown and described, or portions thereof, are not excluded, and it is recognized that various modifications are possible within the scope of the invention. Embodiments of the invention are set forth in the following claims.

The invention will be further described with reference to the examples described herein; however, it is to be understood that the invention is not limited to such examples.

### EXAMPLES

Unless stated otherwise in the Examples, all recombinant DNA techniques are carried out according to standard protocols, for example, as described in Sambrook et al. (2012) Molecular Cloning: A Laboratory Manual, 4th Edition, Cold Spring Harbor Laboratory Press, NY and in Volumes 1 and 2 of Ausubel et al. (1994) Current Protocols in Molecular Biology, Current Protocols, USA. Other references for standard molecular biology techniques include Sambrook and Russell (2001) Molecular Cloning: A Laboratory Manual, Third Edition, Cold Spring Harbor Laboratory Press, NY, Volumes I and II of Brown (1998) Molecular Biology LabFax, Second Edition, Academic Press (UK). Standard materials and methods for polymerase chain reactions can be found in Dieffenbach and Dveksler (1995) PCR Primer: A Laboratory Manual, Cold Spring Harbor Laboratory Press, and in McPherson at al. (2000) PCR—Basics: From Background to Bench, First Edition, Springer Verlag, Germany.

### Example 1: Developing, Making and Validating Exemplary Anti-Human BCMA Antibodies

A BCMA antigen was designed by using the entire extracellular domain of BCMA in fusion to rabbit Fc domain and expressing the recombinant antigen in HEK cells. The antigen was used for immunizations of rabbits and bleeds were later taken to confirm serum reactivity against BCMA in ELISA and IHC. The rabbits showing best immune response against BCMA were chosen for B-cell selection using blood samples from the rabbits.

Briefly, B-cells expressing antibodies binding the immunogen were isolated and cultured monoclonally before testing for specificity in ELISA. ELISA specific clones were further tested in super-sensitive IHC on normal and clinical tissue, both using high and low pH antigen retrieval buffers, respectively. The best performing clones were chosen based on IHC performance.

The antibody variable domains were cloned into a custom-made expression vector based on the pTT5 backbone, containing the constant domains of the heavy and kappal light chain, respectively. Recombinant antibodies were expressed in HEK293-6E cells. The recombinant antibodies were tested for BCMA binding by biolayer interferometry (BLI) on a BLItz, and subsequently tested in IHC by standard FLEX protocols on normal and clinical tissues.

In particular, 3 clones showed specific reactivity against BCMA, and these were tested further, alone and in combinations. The clones 9H5 and 10A2 were the best performing antibodies, and further showed an additive effect in IHC, with the cocktail having higher sensitivity and robustness than the either of the clones alone. The final anti-human BCMA antibody composition is a cocktail of the anti-human BCMA clones 9H5 and 10A2 in an equal ratio.

These antibodies have been tested for specificity on different clinical tissues showing usefulness for in vitro diagnostics by immunohistochemistry.

### BCMA Antibody Clone Development

For the development of anti-human BCMA antibodies 2 antigen designs were used. One for targeting the extracellular domain, and one for targeting the intracellular domain of BCMA. The antigen representing the extracellular domain of BCMA was a recombinant fusion protein containing the entire extracellular domain of human BCMA, amino acids 1-54). The antigen representing the intracellular domain of BCMA was a recombinant fusion protein containing the entire intracellular domain of BCMA, amino acids 78-184. Two groups of rabbits were immunized, one group for each antigen. Serum samples were taken from the rabbits and the titer was tested by ELISA. Subsequently specificity testing was performed using serum sample for IHC of tissue array containing different tissues: Normal tonsils, reactive lymph nodes, normal liver, carcinoid tumor, mamma carcinoma, colon carcinoma, cerebellum, normal prostate, normal kidney, normal pancreas and normal bone marrow, malignant melanoma (clinical tissue), plasmacytoma (clinical tissue) and multiple myeloma (clinical tissue). All rabbits showed specificity against BCMA protein in IHC by varying degree. The rabbit of each antigen group producing the best performing serum sample was chosen for B-cell selection. Blood sample was taken from the rabbit and subjected to B-cell selection, isolating the B-cells producing antibodies binding the BCMA antigen. The isolated B-cells were cultured monoclonally under stimulating conditions, and the resulting cell culture supernatants were tested in ELISA to identify wells containing B-cells producing antigen binding antibodies. ELISA positive cell culture supernatants were further tested in super sensitive IHC, identifying cell culture supernatants having antibodies specific for BCMA in IHC. B-cell selections were performed using rabbits of the extracellular BCMA antigen group yielding 11 B-cell clones showing a high degree of BCMA specificity by IHC. From the group immunized with the intracellular domain B-cell selections yielded 16 clones showing a high degree of BCMA specificity in IHC.

The best of the IHC performing B-cell clones of each of the groups were chosen for cloning. The selection and prioritizing of B-cell clones were performed using the super sensitive IHC on both normal and clinical tissue arrays containing: Normal tonsils, reactive lymph nodes, normal liver, carcinoid tumor, mamma carcinoma, colon carcinoma, cerebellum, normal prostate, normal kidney, normal pancreas and normal bone marrow, malignant melanoma (clinical tissue), and furthermore on BCMA specific positive cell lines (NCI-H929, U-2932 both high BCMA expressing and IM-9 medium BCMA expressing) and BCMA negative cell lines (MCF-07, COLO205).

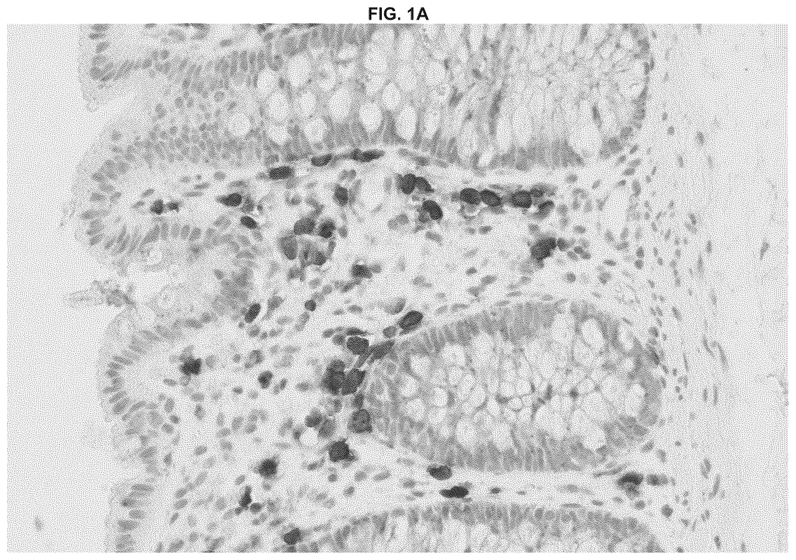

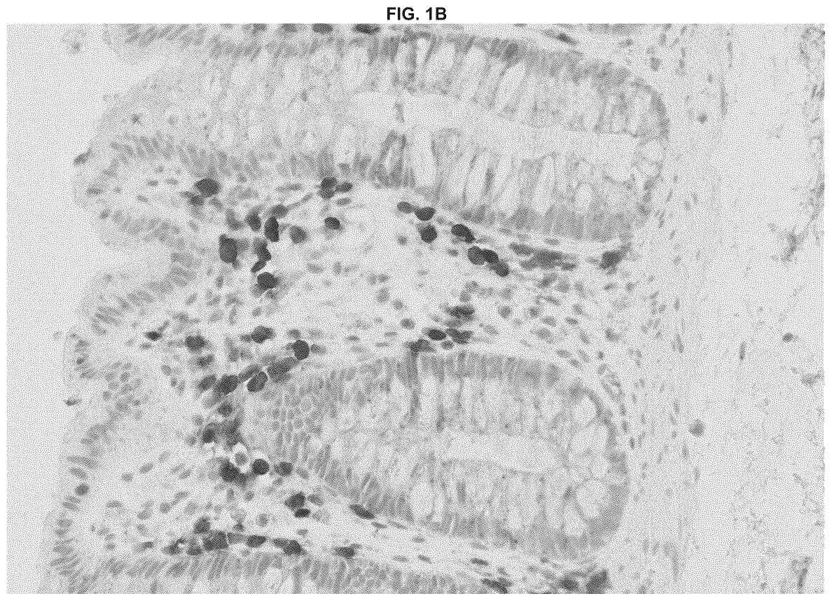

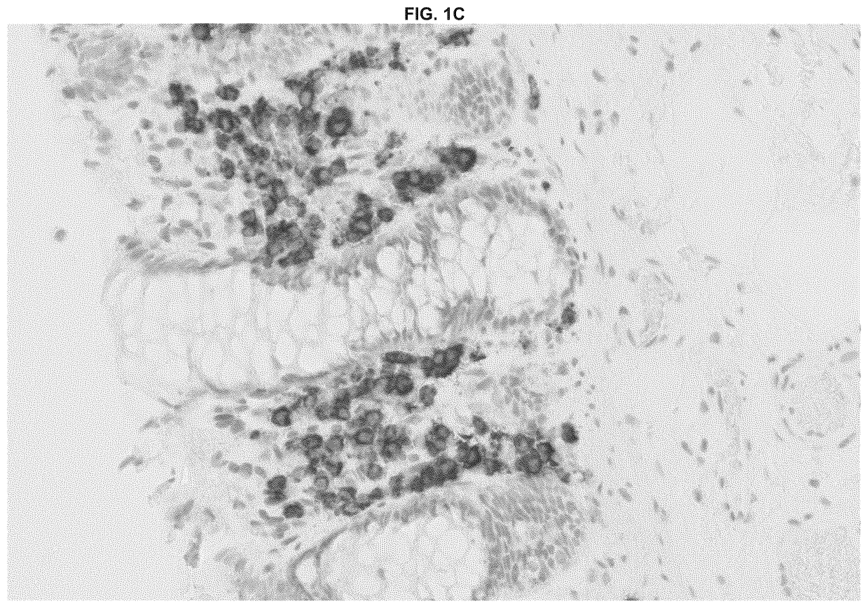

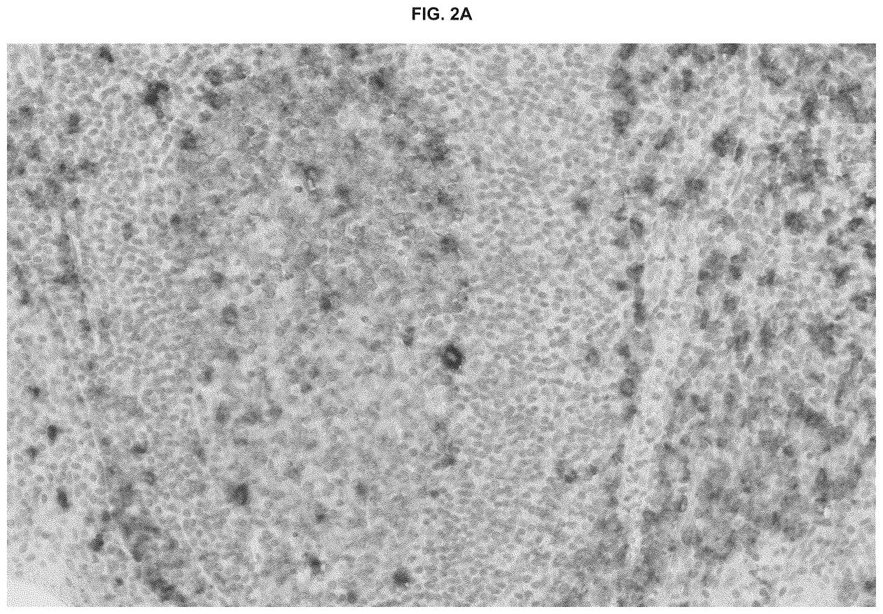

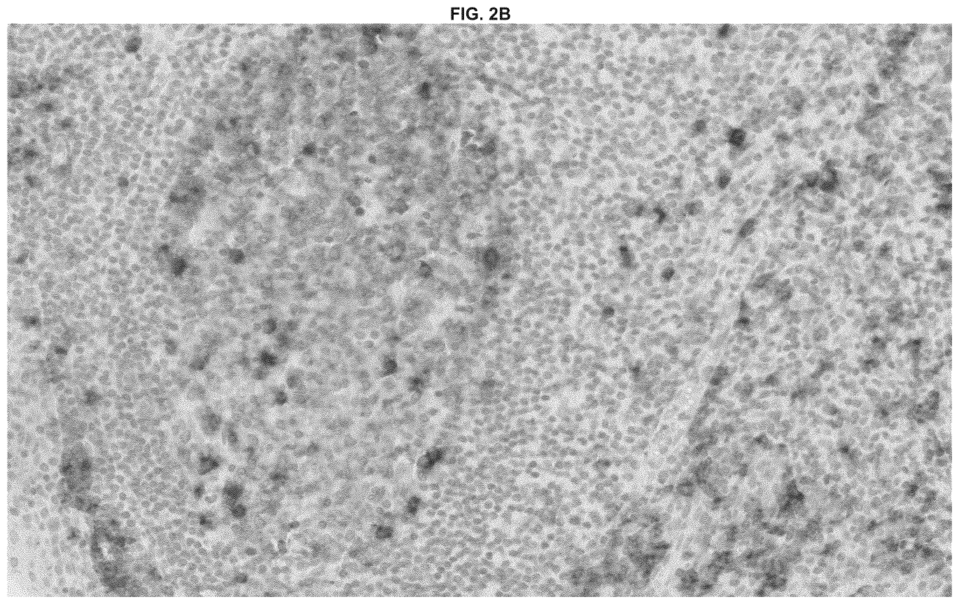

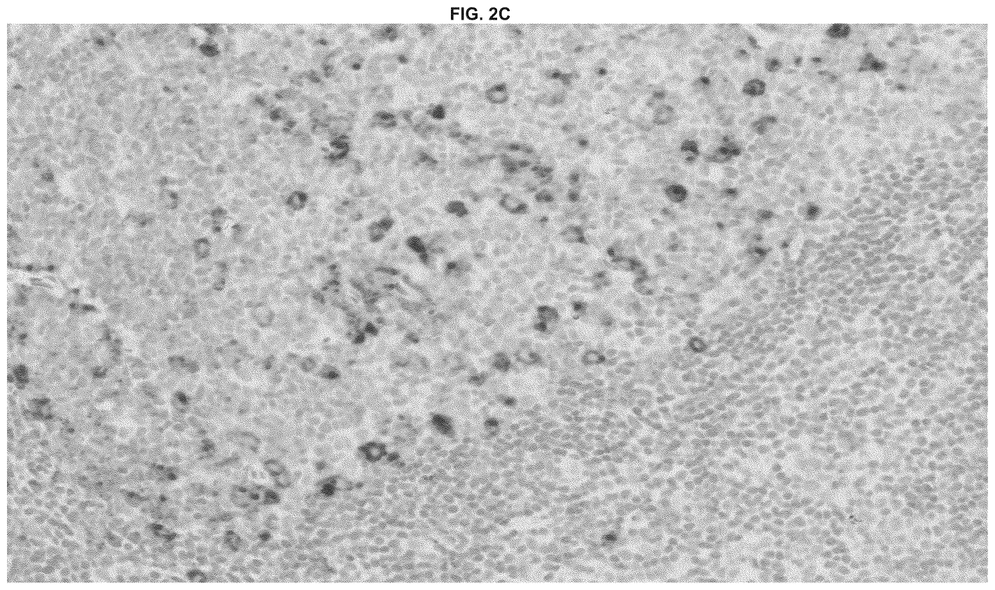

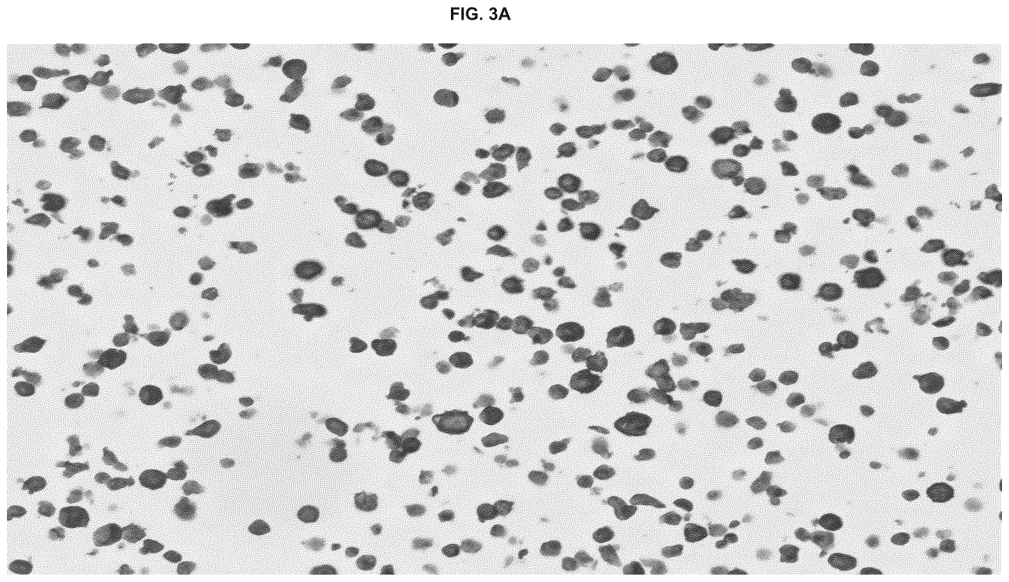

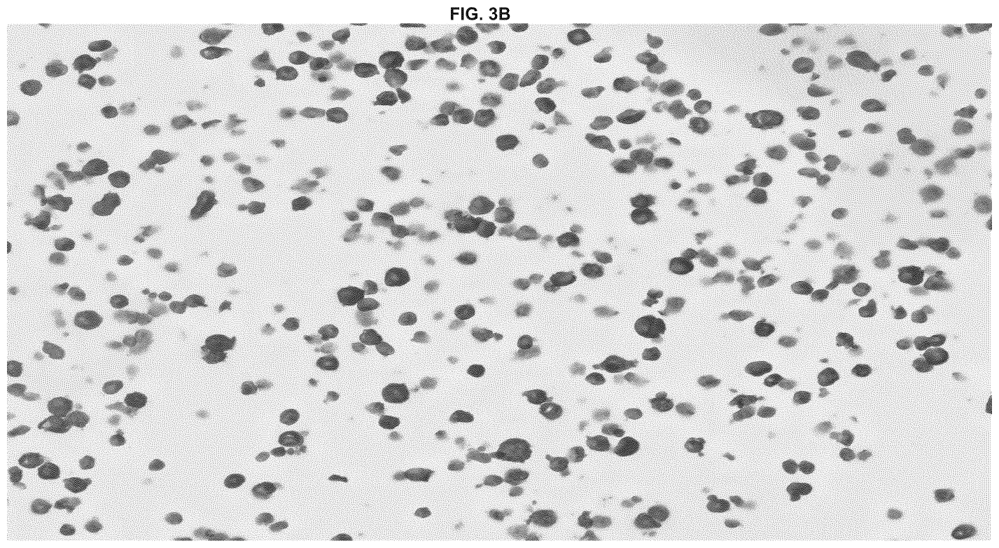

Supersensitive IHC staining of the two best performing B cell clones, 9H5 and 10A2 is illustrated in FIG. 1 (normal colon), FIG. 2 (normal tonsil) and FIG. 3 (BCMA positive cell line U2932).

FIG. 1A (FIG. 1a) illustrates an image of a supersensitive IHC of B cell clone 9H5 on normal colon. Plasma cells in lamina propria of the colon epithelia show membrane and cytoplasmatic staining.

FIG. 1B (FIG. 1b) illustrates an image of a supersensitive IHC of B cell clone 10A2 on normal colon. Plasma cells in lamina propria of the colon epithelia show membrane and cytoplasmatic staining.

FIG. 1C (FIG. 1c) illustrates an image of a supersensitive IHC of B cell clone 5C1 on normal colon. Plasma cells in lamina propria of the colon epithelia show membrane and cytoplasmatic staining.

FIG. 2A (FIG. 2a) illustrates an image of a supersensitive IHC of B cell clone 9H5 on normal tonsil. Germinal center B cells, plasmablasts and plasma cells below and within the tonsillar epithelium (bottom left corner) show membrane and cytoplasmatic staining of different intensity.

FIG. 2B (FIG. 2b) illustrates an image of a supersensitive IHC of B cell clone 10A2 on normal tonsil. Germinal center B cells, plasmablasts and plasma cells below and within the tonsillar epithelium (bottom left corner) show membrane and cytoplasmatic staining of different intensity.

FIG. 2C (FIG. 2c) illustrates an image of a supersensitive IHC of B cell clone 5C1 on normal tonsil. Germinal center B cells, plasmablasts show membrane and cytoplasmatic staining of different intensity.

FIG. 3A (FIG. 3a) illustrates an image of a supersensitive ICH of B cell clone 9H5 on BCMA positive cell line U-2932. Strong to medium membrane staining of virtually all cells, confirming specificity of the BCMA clone 9H5.

FIG. 3B (FIG. 3b) illustrates an image of a supersensitive ICH of B cell clone 10A2 on BCMA positive cell line U-2932. Strong to medium membrane staining of virtually all cells, confirming the specificity of the BCMA clone 10A2.

The monoclonally cultured B-cells of the respective wells of the chosen clones were lysed, and the RNA extracted and used for production of cDNA. The variable heavy and light chains, respectively, were amplified by PCR, using custom made primers and the PCR products were cloned into expression vectors (based on pTT5 backbone) containing the respective rabbit constant heavy and light IgG chains, yielding functional antibody coding sequences. Unique functional antibody coding sequences were identified by sequence analysis of the cloned heavy and light chains, respectively. The heavy and light chains plasmids were transfected into HEK293-6E cell line and recombinant antibody was produced and tested for binding to the antigen by biolayer interferometry (BLI) on a BLItz. Antibodies showing antigen specificity were subsequently tested in standard IHC (Envision FLEX™ (Dako Omnis)) protocol on normal and clinical tissues, consisting of the above mentioned normal clinical tissue arrays and BCMA positive and negative cell lines plus single clinical tissues consisting of: Plasmacytoma, multiple myeloma, Burkitt lymphoma and Hodgkin lymphoma.

In particular, two clones targeting the extracellular domain of BCMA, clone 9H5 and 10A2, and one clone targeting the intracellular domain, clone 5C1, showed superior IHC performance. These 2 clones recognizing the extracellular domain of BCMA showed minor differences in IHC performance. The specificity of these clones were confirmed by testing the above mentioned BCMA specific cell lines and negative cell lines (see FIGS. 3a, 3b, 10 and 11). Furthermore, the IHC expression of the clones were compared to other known antibodies targeting plasma cells (CD138), B- and T-cells (CD30, CD5) and T-cells exclusively (CD3).

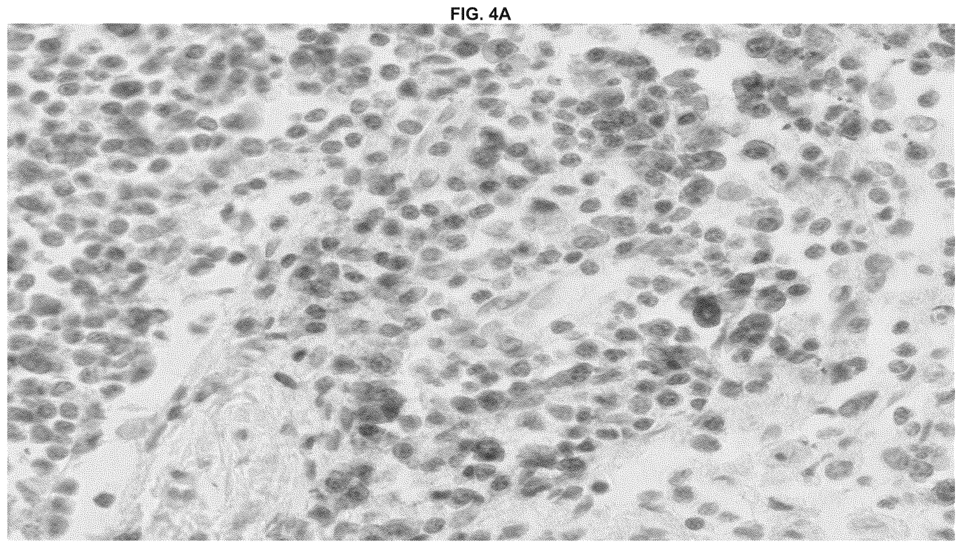

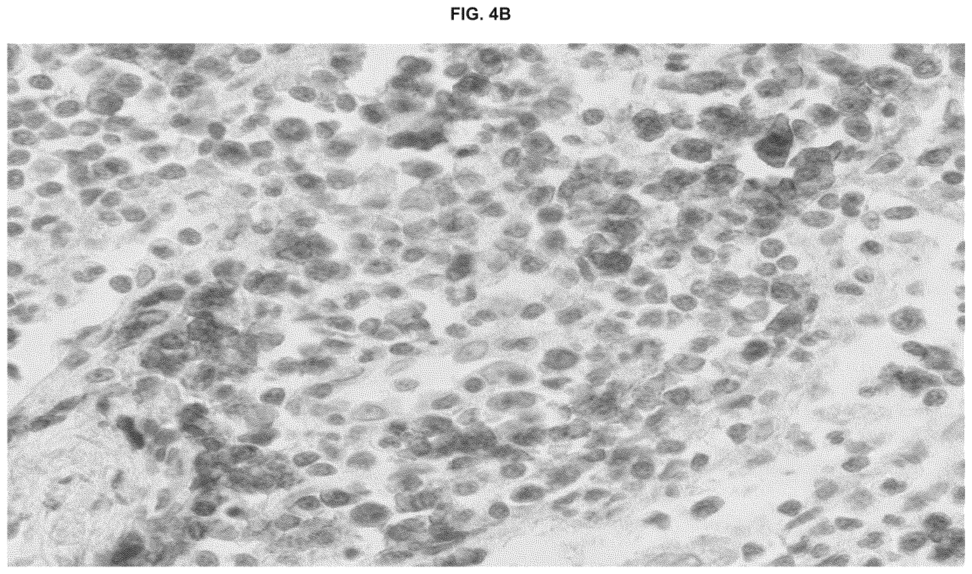

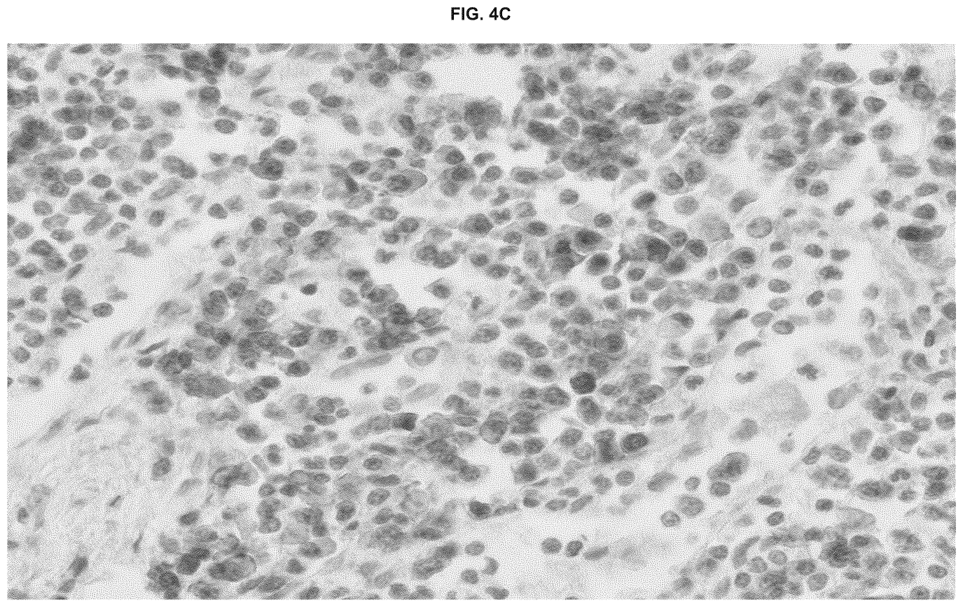

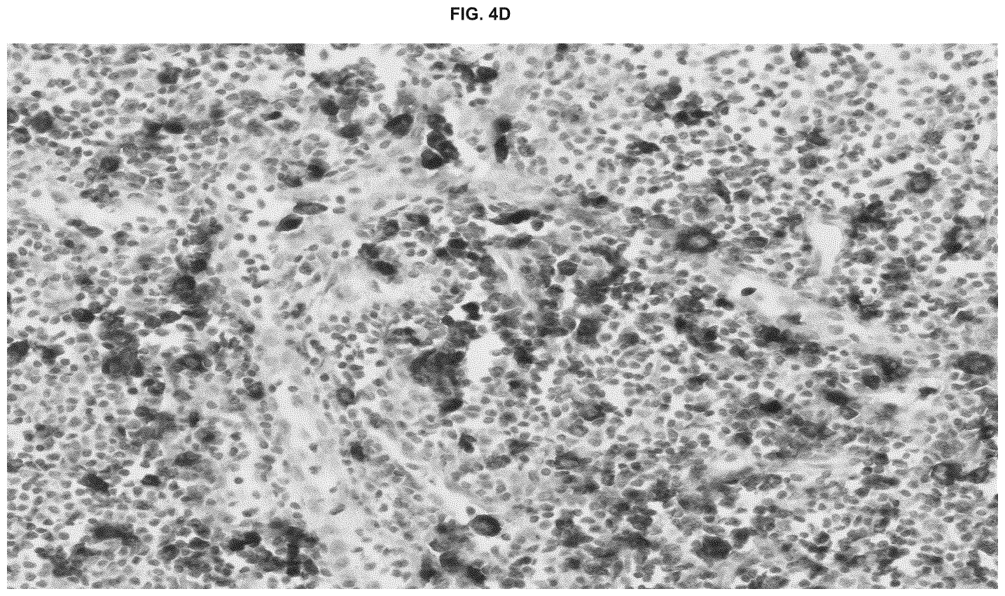

Finally, the IHC expression and specificity of BCMA clones 5C1, 9H5 and 10A2 were evaluated and approved by a pathologist. Tests was made mixing the extracellular clones 9H5 and 10A2 for IHC. The mix showed an additive effect (stronger or equal intensity and more distinct staining with lower antibody concentration) of the respective antibodies indicating that the epitopes are different for the two clones 9H5 and 10A2. The optimal antibody concentration of the mix was determined in IHC titration to be 0.25× the antibody concentration of either clone alone (0.25×9H5+0.25×10A2). Standard IHC (Envision FLEX™) of 9H5, 10A2 and of supersensitive IHC of the extracellular clone 5C1 separately and a mix of 9H5 and 10A2 clones in lower concentrations (standard IHC) is illustrated in FIGS. 4 to 6 on lymph node tissue, plasmacytoma and multiple myeloma tissue respectively (multiple myeloma not shown for clone 5C1 in these figures).

FIG. 4A (FIG. 4a) illustrates an image of a standard IHC (Envision FLEX™) of clone 9H5 alone on reactive lymph node. Moderate cytoplasmatic and membrane staining of plasma cells.

FIG. 4B (FIG. 4b) illustrates an image of a standard IHC (Envision FLEX™) of clone 10A2 alone on reactive lymph node. Strong to moderate cytoplasmatic and membrane staining of plasma cells.

FIG. 4C (FIG. 4c) illustrates an image of a standard IHC (Envision FLEX™) of mix (0.25 antibody concentration) of clone 9H5 and 10A2 on reactive lymph node. Strong to moderate, distinct cytoplasmatic and membrane staining of plasma cells.

FIG. 4D (FIG. 4d) illustrates an image of a supersensitive IHC of clone 5C1 on reactive lymph node. Strong cytoplasmatic and membrane staining of plasma cells.

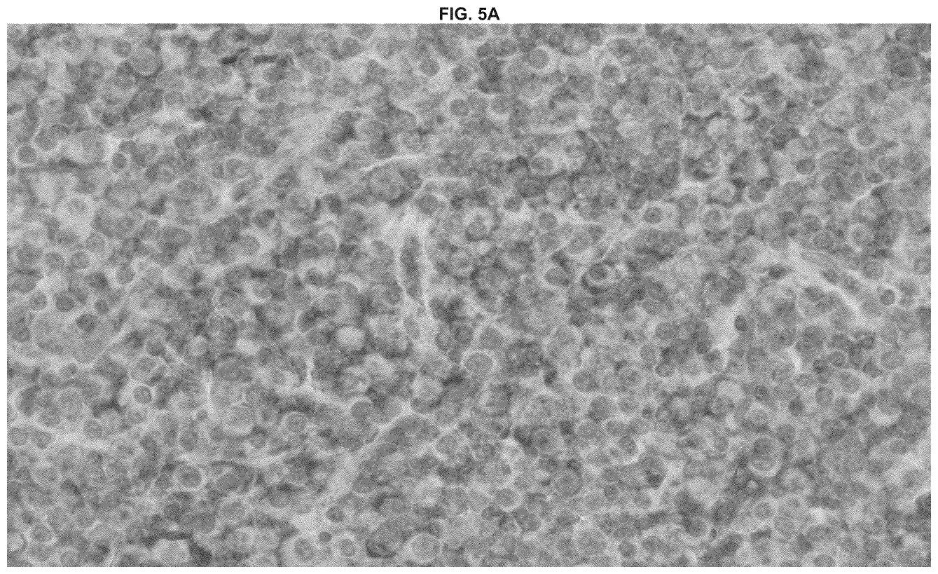

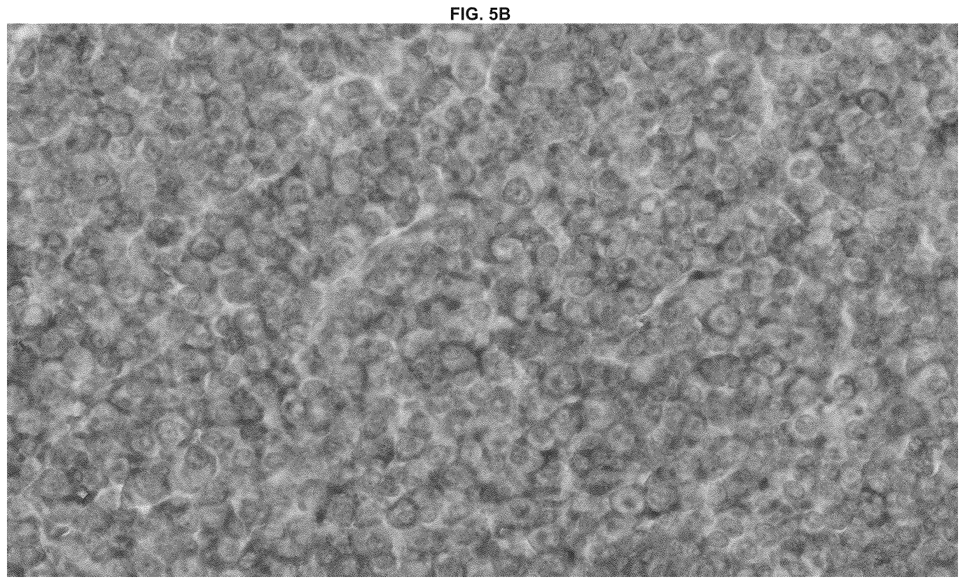

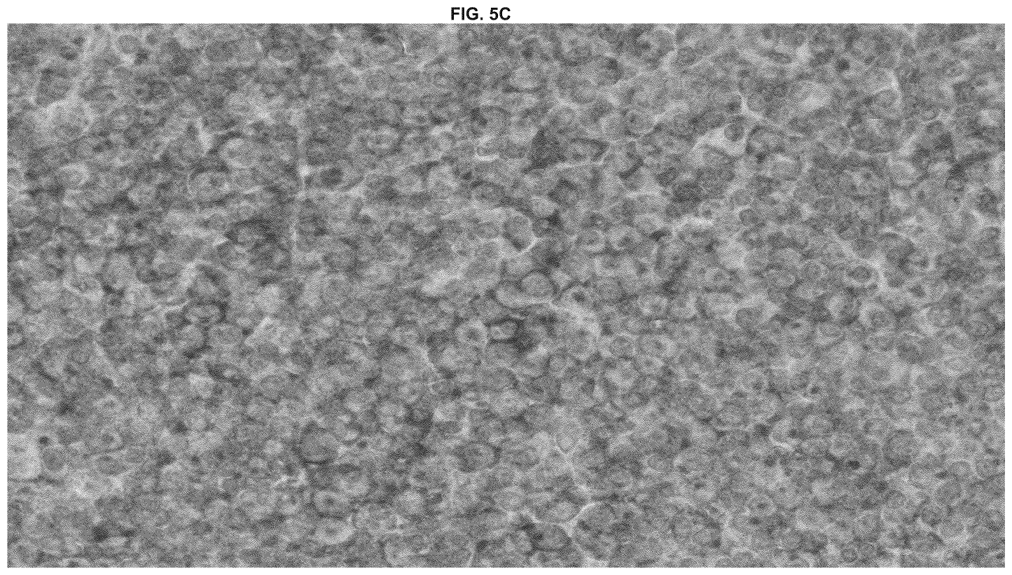

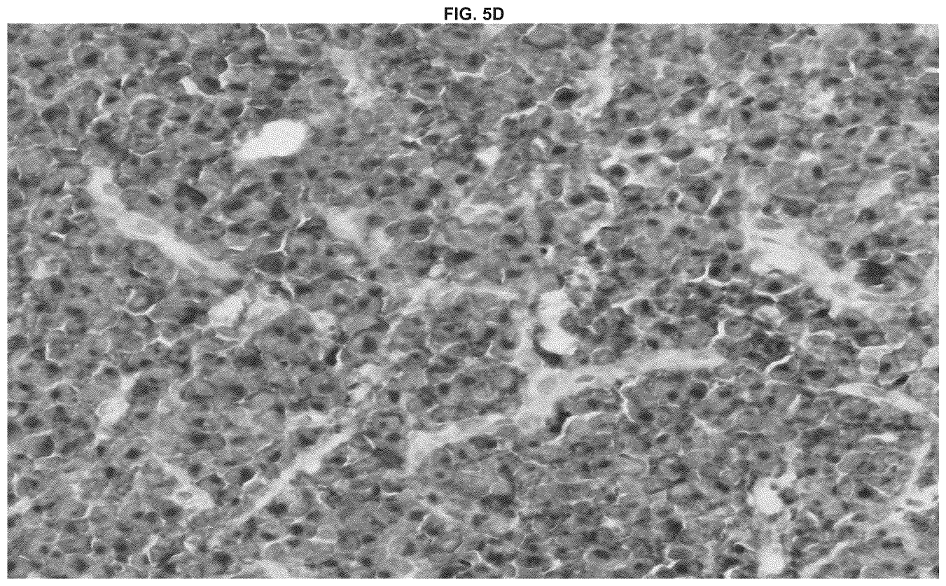

FIG. 5A (FIG. 5a) illustrates an image of a standard IHC (Envision FLEX™) of clone 9H5 alone on plasmacytoma. Strong to moderate, primarily membrane and few Golgi staining of plasmacytoma tumor cells.

FIG. 5B (FIG. 5b) illustrates an image of a standard IHC (Envision FLEX™) of clone 10A2 alone on plasmacytoma. Strong primarily membrane and some Golgi staining of plasmacytoma tumor cells.

FIG. 5C (FIG. 5c) illustrates an image of a standard IHC (Envision FLEX™) of mix (0.25 antibody concentration) of clone 9H5 and 10A2 on plasmacytoma. Strong and distinct, primarily membrane and some Golgi staining of plasmacytoma tumor cells.

FIG. 5D (FIG. 5d) illustrates an image of a supersensitive IHC of clone 5C1 on plasmacytoma. Strong to moderate, membrane and many strong Golgi staining of plasmacytoma tumor cells.

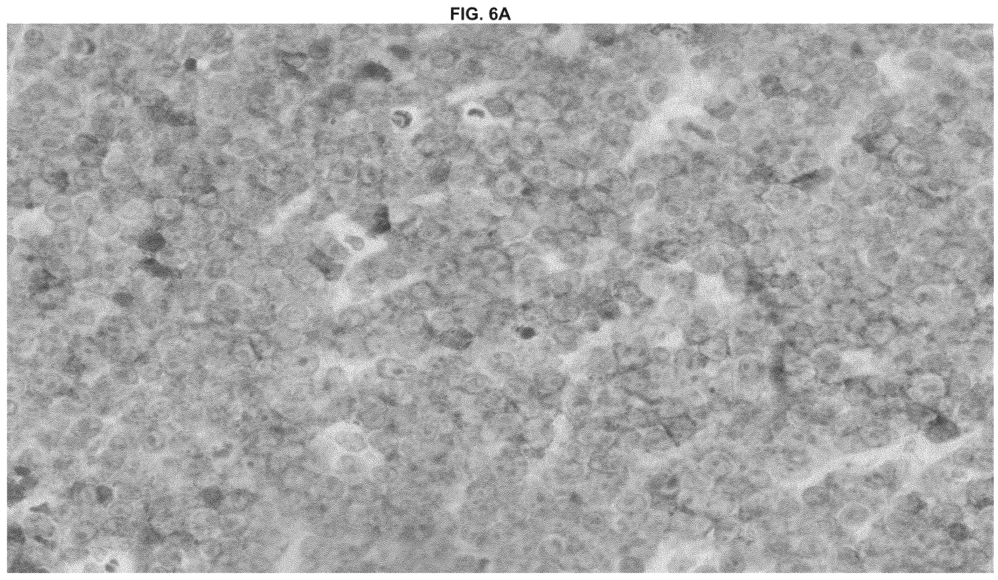

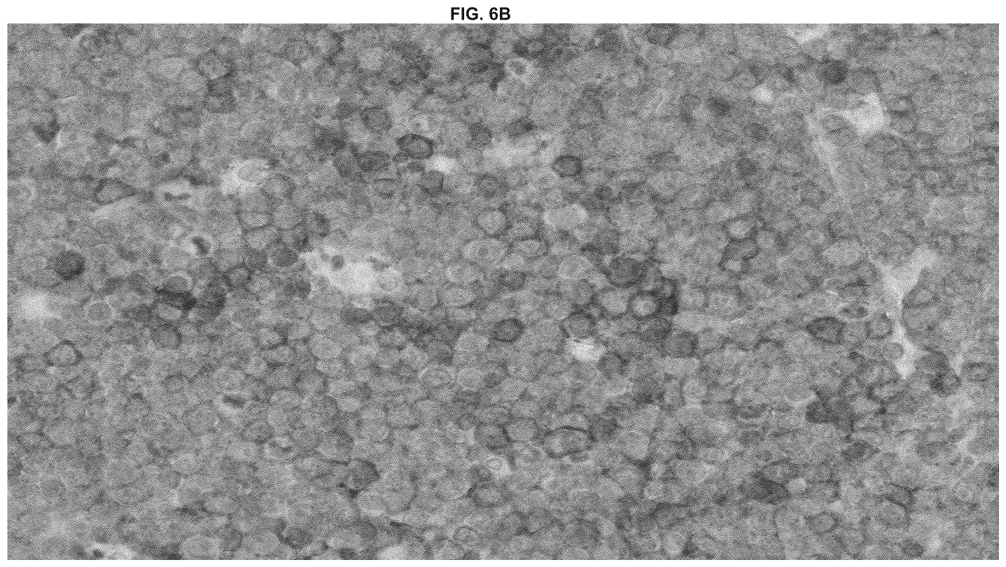

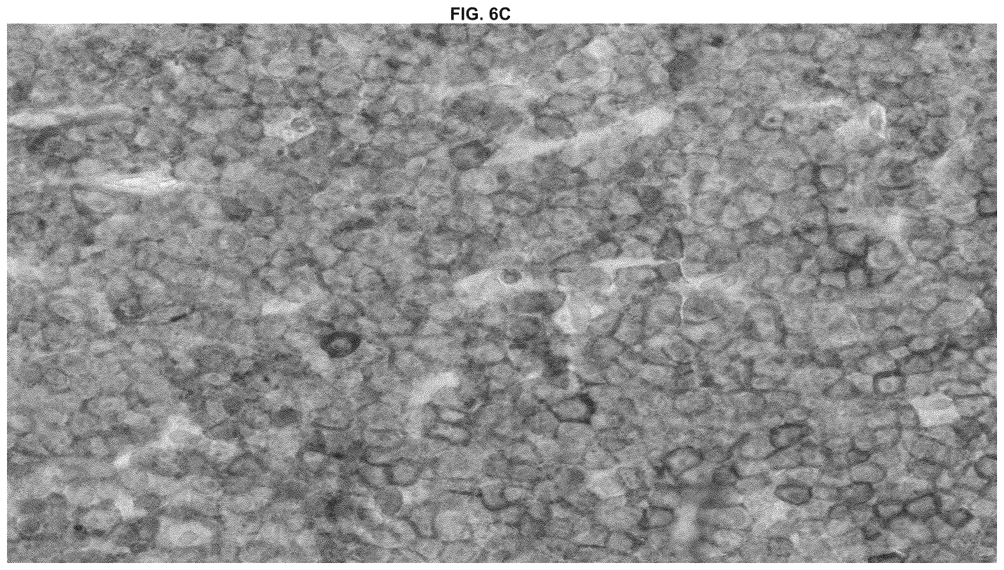

FIG. 6A (FIG. 6a) illustrates an image of a standard IHC (Envision FLEX™) of clone 9H5 alone on multiple myeloma. Strong to moderate, primarily membrane staining of myeloma tumor cells.

FIG. 6B (FIG. 6b) illustrates an image of a standard IHC (Envision FLEX™) of clone 10A2 alone on multiple myeloma. Strong primarily membrane staining of myeloma tumor cells.

FIG. 6C (FIG. 6c) illustrates an image of a standard IHC (Envision FLEX™) of mix (0.25 antibody concentration) of clone 9H5 and 10A2 on multiple myeloma. Strong and distinct, primarily membrane staining of myeloma tumor cells.

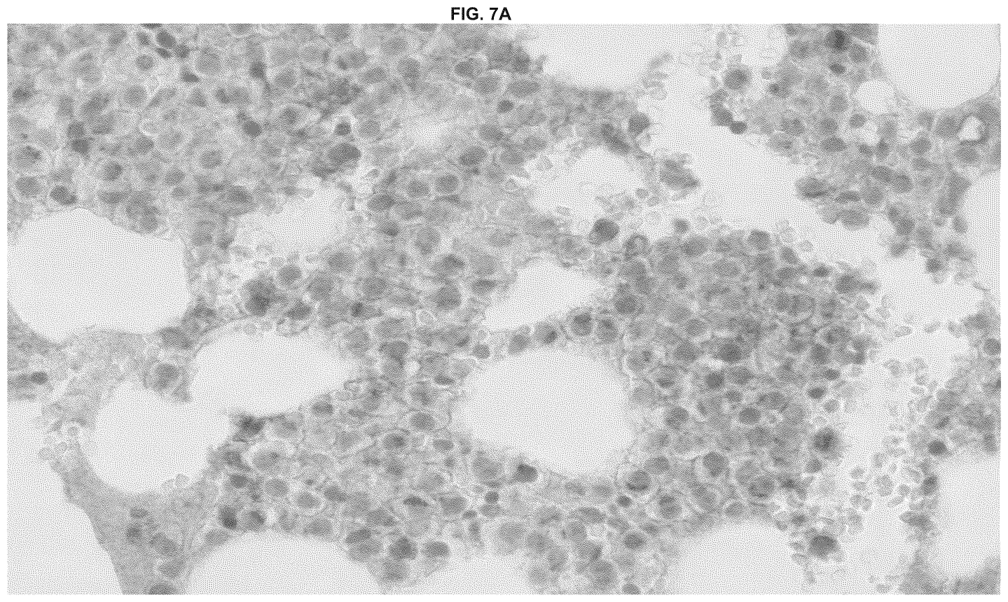

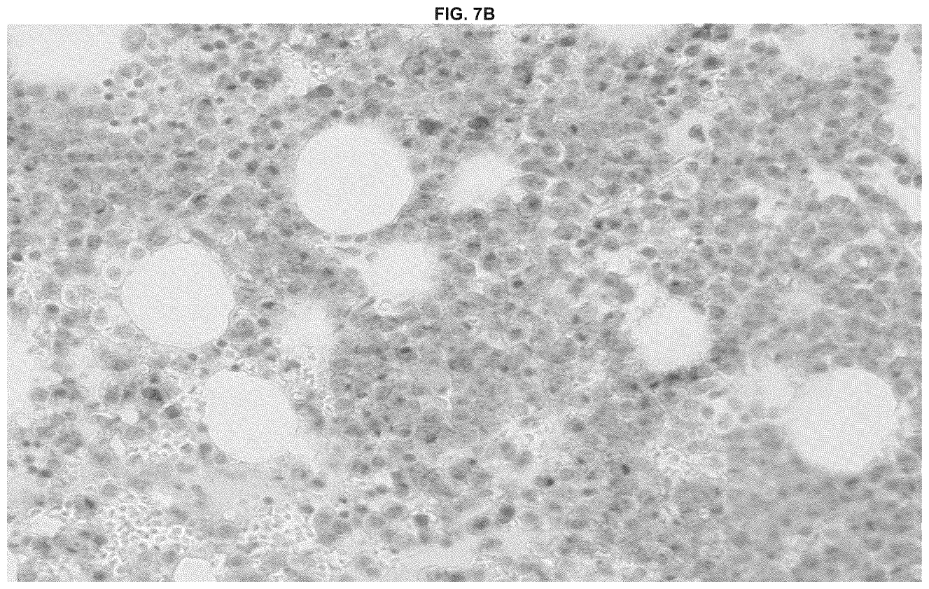

Further tests were made to determine the effect of mixing the 2 extracellular antibody clones 9H5 and 10A2. An example of an optimal protocol of the mix of the two antibodies in standard IHC (EnVision FLEX™) and of the intracellular clone 5C1 alone, is illustrated in FIGS. 7-11 on multiple myeloma, plasmacytoma, Burkitt lymphoma and positive and negative cell line.

FIG. 7A (FIG. 7a) illustrates an image of a standard IHC optimal protocol of mix of clone 9H5 and 10A2 on multiple myeloma. Distinct membrane and Golgi staining of myeloma tumour cells.

FIG. 7B (FIG. 7b) illustrates an image of a standard IHC optimal protocol of clone 5C1 on multiple myeloma. Distinct membrane and Golgi staining of myeloma tumour cells.

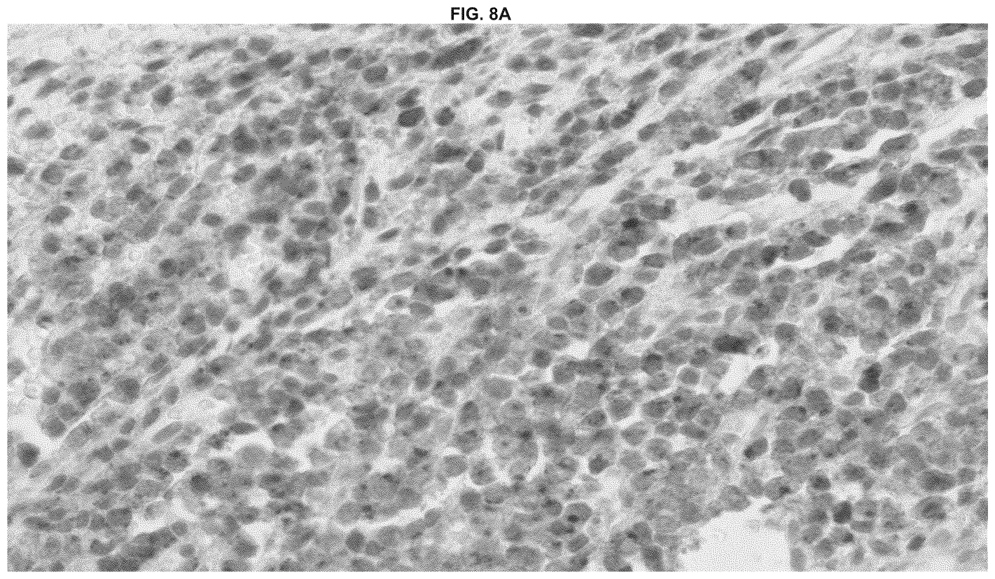

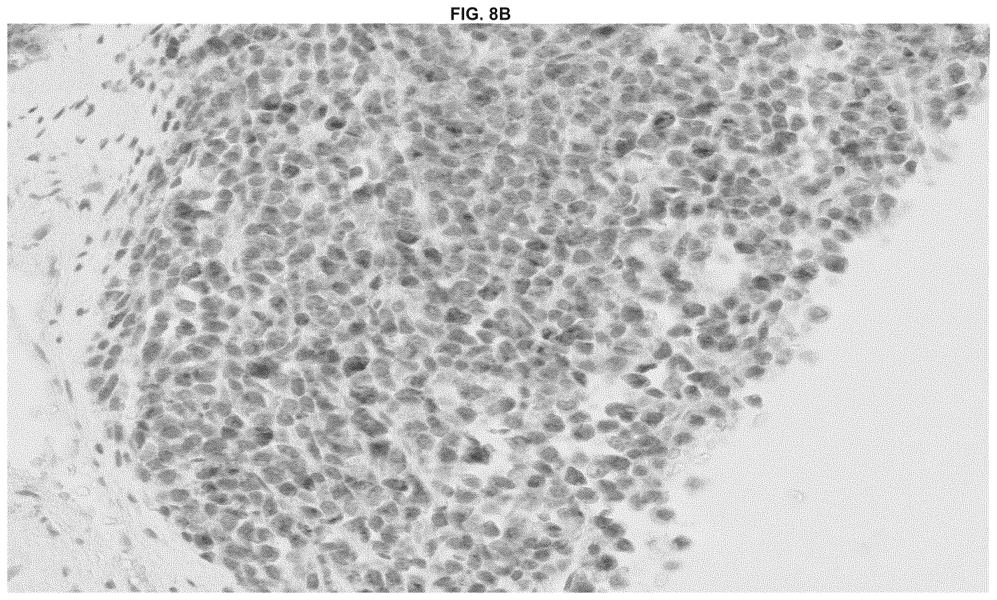

FIG. 8A (FIG. 8a) illustrates an image of a standard IHC optimal protocol of mix of clone 9H5 and 10A2 on plasmacytoma. Distinct membrane and strong Golgi staining of plasmacytoma tumour cells.

FIG. 8B (FIG. 8b) illustrates an image of a standard IHC optimal protocol of clone 5C1 on plasmacytoma. Moderate cytoplasmatic, a few membrane and strong Golgi staining of plasmacytoma tumour cells.

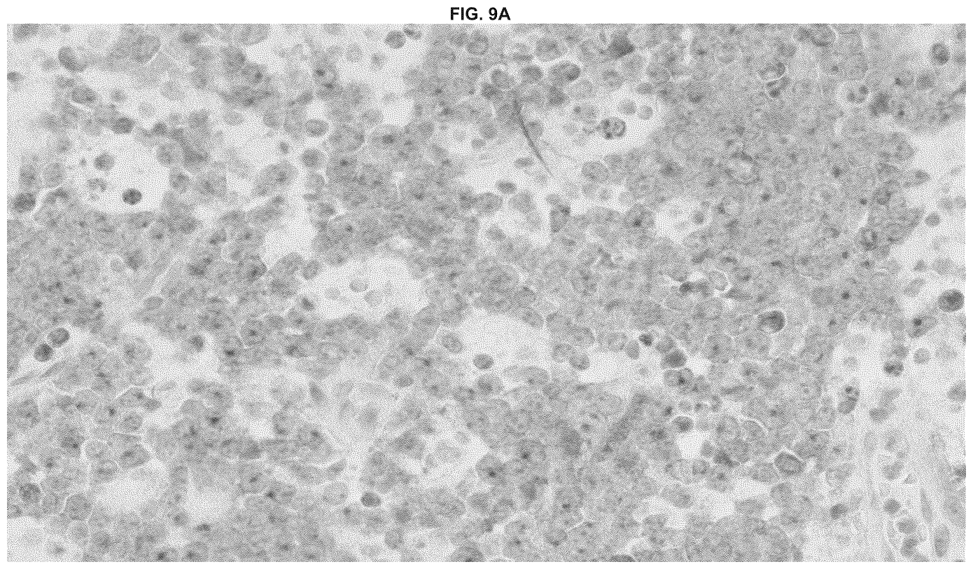

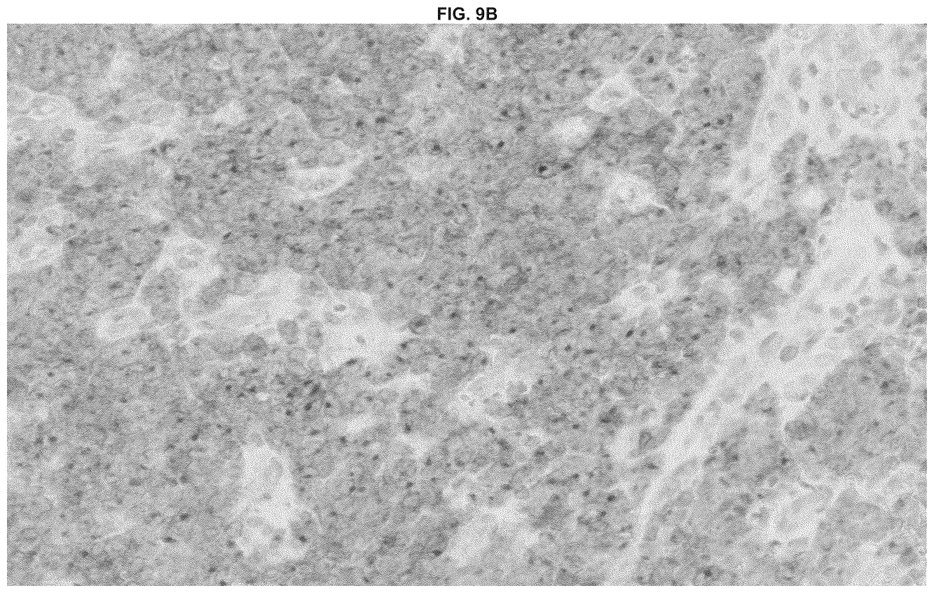

FIG. 9A (FIG. 9a) illustrates an image of a standard IHC optimal protocol of mix of clone 9H5 and 10A2 on Burkitt lymphoma. Distinct membrane and Golgi staining of tumour cells.

FIG. 9B (FIG. 9b) illustrates an image of a standard IHC optimal protocol of clone 5C1 on Burkitt lymphoma. Distinct membrane and Golgi staining of tumour cells.

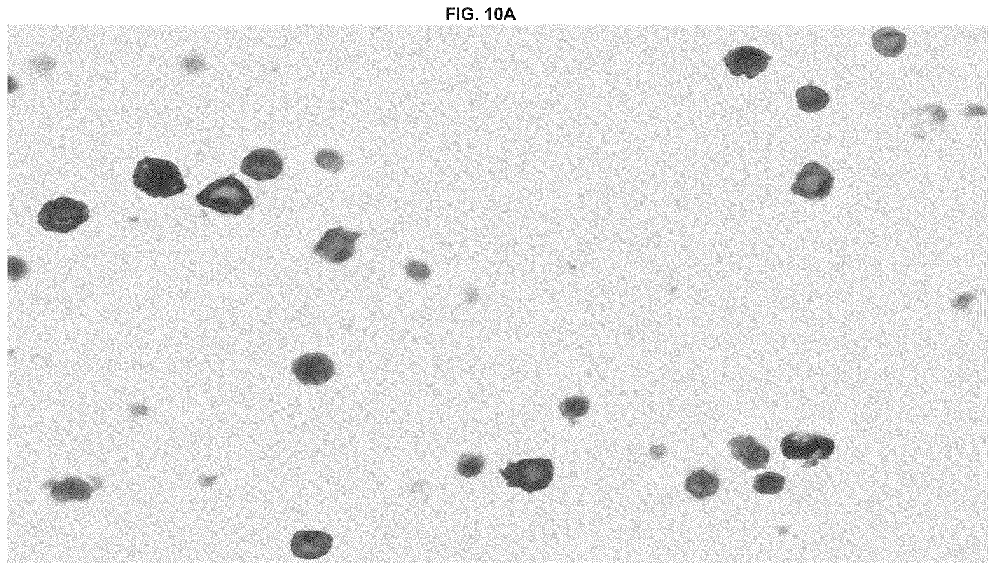

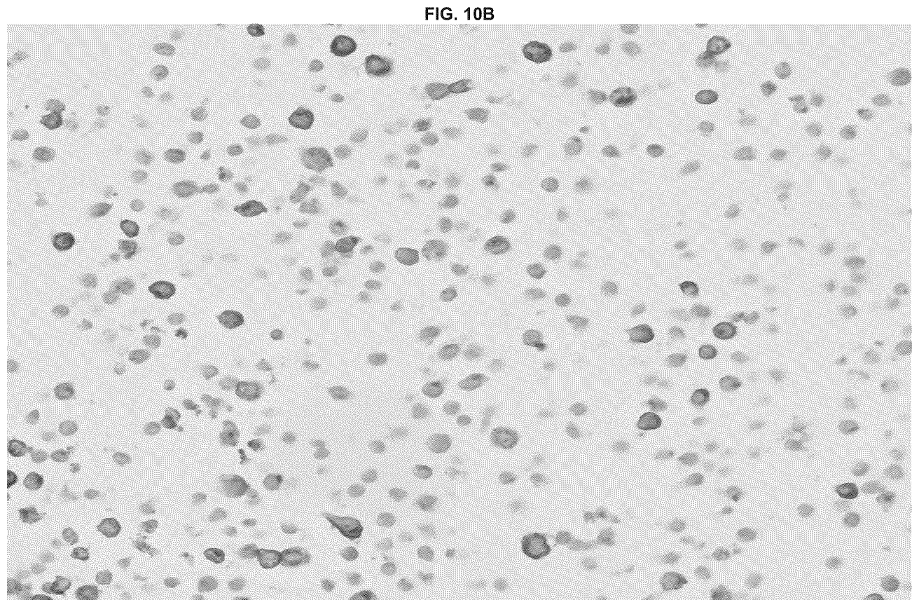

FIG. 10A (FIG. 10a) illustrates an image of a standard IHC optimal protocol of mix of clone 9H5 and 10A2 on BCMA positive high expressing cell line NCI-H929.

FIG. 10B (FIG. 10v) illustrates an image of a standard IHC optimal protocol of clone 5C1 on BCMA positive high expressing cell line U-2932.

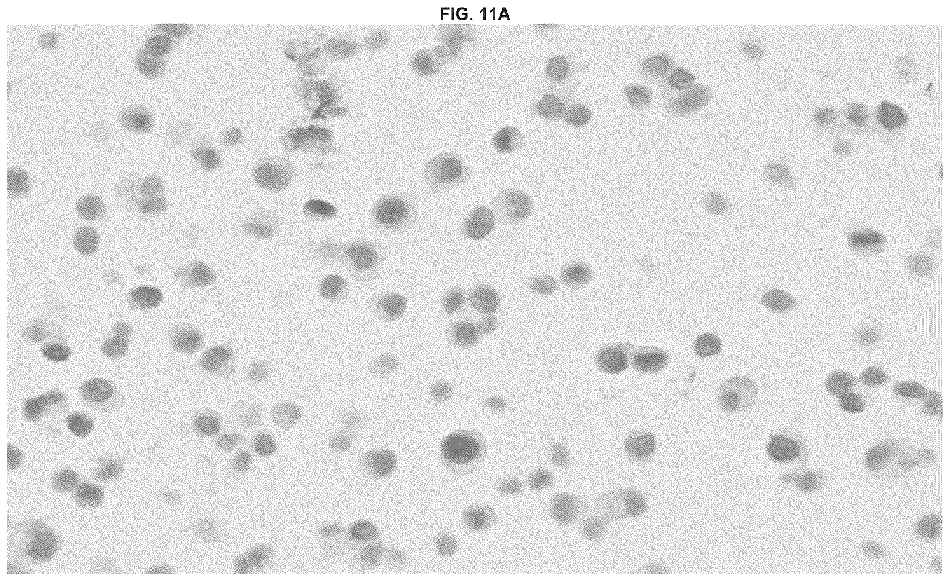

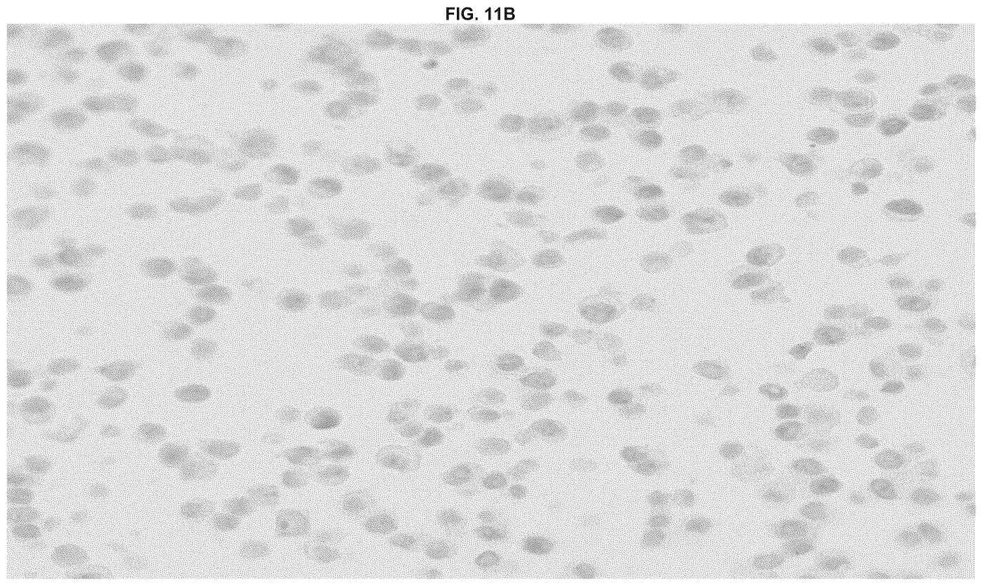

FIG. 11A (FIG. 11a) illustrates an image of a standard IHC optimal protocol of mix of clone 9H5 and 10A2 on BCMA negative cell line MCF-7.

FIG. 11B (FIG. 11b) illustrates an image of a standard IHC optimal protocol of clone 5C1 on BCMA negative cell line MCF-7.

BCMA antibody clone sequences
Constant domains (of all 3 clones)
Constant (Kappa1):
(SEQ ID NO: 7)
GDPVAPTVLIFPPAADQVATGTVTIVCVANKYFPDVTVTWEVDGTTQTTGIENSK
TPQNSADCTYNLSSTLTLTSTQYNSHKEYTCKVTQGTTSVVQSFNRGDC
VH (IgG):
(SEQ ID NO: 8)
GQPKAPSVFPLAPCCGDTPSSTVTLGCLVKGYLPEPVTVTWNSGTLTNGVRTFPS
VRQSSGLYSLSSVVSVTSSSQPVTCNVAHPATNTKVDKTVAPSTCSKPTCPPPELL
GGPSVFIFPPKPKDTLMISRTPEVTCVVVDVSQDDPEVQFTWYINNEQVRTARPP
LREQQFNSTIRVVSTLPIAHQDWLRGKEFKCKVHNKALPAPIEKTISKARGQPLEP
KVYTMGPPREELSSRSVSLTCMINGFYPSDISVEWEKNGKAEDNYKTTPAVLDS
DGSYFLYSKLSVPTSEWQRGDVFTCSVMHEALHNHYTQKSISRSPGK
Clone 5C1 (intracellular)
VH
(SEQ ID NO: 1)
QSLEESRGGLFKPTDTLTLTCTVSGFSLKTYVINWVRQAPGNGLEWIGIIDTGDM
TYYASWAKSRSTITRNTNENTVTLKMTSLTAADTATYFCASGDIWGPGTLVTVS
S
CDR1: a.a. 25-32 (GFSLKTYV); CDR2: a.a. 50-56 (IDTGDMT); CDR3: a.a. 95-99
(ASGDI). CDR numbering according to IMGT (www.imgt.org)
Full length heavy chain, including signal peptide
(SEQ ID NO: 15)
METGLRWLLLVAVLKGVQCQSLEESRGGLFKPTDTLTLTCTVSGFSLKTYVINW
VRQAPGNGLEWIGIIDTGDMTYYASWAKSRSTITRNTNENTVTLKMTSLTAADT
ATYFCASGDIWGPGTLVTVSSGQPKAPSVFPLAPCCGDTPSSTVTLGCLVKGYLP
EPVTVTWNSGTLTNGVRTFPSVRQSSGLYSLSSVVSVTSSSQPVTCNVAHPATNT
KVDKTVAPSTCSKPTCPPPELLGGPSVFIFPPKPKDTLMISRTPEVTCVVVDVSQD
DPEVQFTWYINNEQVRTARPPLREQQFNSTIRVVSTLPIAHQDWLRGKEFKCKVH
NKALPAPIEKTISKARGQPLEPKVYTMGPPREELSSRSVSLTCMINGFYPSDISVE
WEKNGKAEDNYKTTPAVLDSDGSYFLYSKLSVPTSEWQRGDVFTCSVMHEALH
NHYTQKSISRSPGK
VL
(SEQ ID NO: 2)
AQVLTQTASPVSAAVGGTVTINCQASQSVYSNWLSWFQQKPGQPPKRLIYSAST
LASGVSSRFKGSGSGTQFTLTISDVQCDDAATYYCLGEFNCARADCRAFGGGTE
VVVK
CDR1: a.a. 27-33 (QSVYSNW); CDR2: a.a. 51-53 (SAS); CDR3: a.a. 90-102
(LGEFNCARADCRA). CDR numbering according to IMGT (www.imgt.org)
Full length light chain, including signal peptide
(SEQ ID NO: 16)
MDTRAPTQLLGLLLLWLPGATFAQVLTQTASPVSAAVGGTVTINCQASQSVYSN
WLSWFQQKPGQPPKRLIYSASTLASGVSSRFKGSGSGTQFTLTISDVQCDDAATY
YCLGEFNCARADCRAFGGGTEVVVKGDPVAPTVLIFPPAADQVATGTVTIVCVA
NKYFPDVTVTWEVDGTTQTTGIENSKTPQNSADCTYNLSSTLTLTSTQYNSHKEY
TCKVTQGTTSVVQSFNRGDC
Clone 9H5 (Extracellular)
VH
(SEQ ID NO: 3)
QSLEESGGRLVTPGGSLTLTCTVSGFSLSAYAMSWVRQAPGKGLEWIGYINYDGI
AYYTNWAKGRFTISKTSTTVDLKITSPTTEDTAAYFCASDLLGVFNLWGQGTLV
TVSS
CDR1: a.a. 25-32 (GFSLSAYA); CDR2: a.a. 50-56 (INYDGIA); CDR3: a.a. 93-102
(ASDLLGVFNL). CDR numbering according to IMGT (www.imgt.org)
Full length heavy chain, including signal peptide
(SEQ ID NO: 17)
METGLRWLLLVAVLKGVQCQSLEESGGRLVTPGGSLTLTCTVSGFSLSAYAMS
WVRQAPGKGLEWIGYINYDGIAYYTNWAKGRFTISKTSTTVDLKITSPTTEDTAA
YFCASDLLGVFNLWGQGTLVTVSSGQPKAPSVFPLAPCCGDTPSSTVTLGCLVK
GYLPEPVTVTWNSGTLTNGVRTFPSVRQSSGLYSLSSVVSVTSSSQPVTCNVAHP
ATNTKVDKTVAPSTCSKPTCPPPELLGGPSVFIFPPKPKDTLMISRTPEVTCVVVD
VSQDDPEVQFTWYINNEQVRTARPPLREQQFNSTIRVVSTLPIAHQDWLRGKEFK
CKVHNKALPAPIEKTISKARGQPLEPKVYTMGPPREELSSRSVSLTCMINGFYPSD
ISVEWEKNGKAEDNYKTTPAVLDSDGSYFLYSKLSVPTSEWQRGDVFTCSVMHE
ALHNHYTQKSISRSPGK
VL
(SEQ ID NO: 4)
AAVLTQTASPVSAAVGGTVTINCQASQSVYSNNRLSWFQQKPGQPPKLLIYYAS
FLASGVPSRFKGSGSGAQFTLTISDLQCDDAATYYCAGGYISYSDNAFGGGTEVV
VK
CDR1: a.a. 27-34 (QSVYSNNR); CDR2: a.a. 52-54 (YAS); CDR3: a.a. 91-101
(AGGYISYSDNA). CDR numbering according to IMGT (www.imgt.org)
Full length light chain, including signal peptide
(SEQ ID NO: 18)
MDTRAPTQLLGLLLLWLPGAAFAAVLTQTASPVSAAVGGTVTINCQASQSVYSN
NRLSWFQQKPGQPPKLLIYYASFLASGVPSRFKGSGSGAQFTLTISDLQCDDAAT
YYCAGGYISYSDNAFGGGTEVVVKGDPVAPTVLIFPPAADQVATGTVTIVCVAN
KYFPDVTVTWEVDGTTQTTGIENSKTPQNSADCTYNLSSTLTLTSTQYNSHKEYT
CKVTQGTTSVVQSFNRGDC
Clone 10A2 (Extracellular)
VH
(SEQ ID NO: 5)
QSVEESGGRLVTPGTPLTLTCTASGFSLTTYAMGWVRQAPGKGLEWIGYIWSSG
TTDYASWAKGRFTISKTSPTVDLKMTSPTTEDTATYFCARYINYVTGDLWGQGT
LVTVSS
CDR1: a.a. 25-32 (GFSLTTYA); CDR2: a.a. 50-56 (IWSSGTT); CDR3: a.a. 93-103
(ARYINYVTGDL). CDR numbering according to IMGT (www.imgt.org)
(SEQ ID NO: 19)
Full length heavy chain, including signal peptide
METGLRWLLLVAVLKGVQCQSVEESGGRLVTPGTPLTLTCTASGFSLTTYAMG
WVRQAPGKGLEWIGYIWSSGTTDYASWAKGRFTISKTSPTVDLKMTSPTTEDTA
TYFCARYINYVTGDLWGQGTLVTVSSGQPKAPSVFPLAPCCGDTPSSTVTLGCLV
KGYLPEPVTVTWNSGTLTNGVRTFPSVRQSSGLYSLSSVVSVTSSSQPVTCNVAH
PATNTKVDKTVAPSTCSKPTCPPPELLGGPSVFIFPPKPKDTLMISRTPEVTCVVV
DVSQDDPEVQFTWYINNEQVRTARPPLREQQFNSTIRVVSTLPIAHQDWLRGKEF
KCKVHNKALPAPIEKTISKARGQPLEPKVYTMGPPREELSSRSVSLTCMINGFYPS
DISVEWEKNGKAEDNYKTTPAVLDSDGSYFLYSKLSVPTSEWQRGDVFTCSVM
HEALHNHYTQKSISRSPGK
VL
(SEQ ID NO: 6)
AQVLTQTPSPVSADVGGTVTINCQASQSVYNNVLLAWYQQKAGQPPKLLIYKAS
TLASGVSSRFKGSGSGTQFTLTISGVQCDDAATYYCQGEFSCSSADCTAFGGGTE
VVVK
CDR1: a.a. 27-34 (QSVYNNVL); CDR2: a.a. 52-54 (KAS); CDR3: a.a. 91-103
(QGEFSCSSADCTA). CDR numbering according to IMGT (www.imgt.org)
Full length light chain, including signal peptide
(SEQ ID NO: 20)
MDTRAPTQLLGLLLLWLPGATFAQVLTQTPSPVSADVGGTVTINCQASQSVYNN
VLLAWYQQKAGQPPKLLIYKASTLASGVSSRFKGSGSGTQFTLTISGVQCDDAA
TYYCQGEFSCSSADCTAFGGGTEVVVKGDPVAPTVLIFPPAADQVATGTVTIVCV
ANKYFPDVTVTWEVDGTTQTTGIENSKTPQNSADCTYNLSSTLTLTSTQYNSHKE
YTCKVTQGTTSVVQSFNRGDC

### REFERENCES

1. Ng, L. G., et al., B cell-activating factor belonging to the TNF family (BAFF)-R is the principal BAFF receptor facilitating BAFF costimulation of circulating T and B cells. J Immunol, 2004. 173(2): p. 807-17.
2. Carpenter, R. O., et al., B-cell maturation antigen is a promising target for adoptive T-cell therapy of multiple myeloma. Clin Cancer Res, 2013. 19(8): p. 2048-60.
3. Ali, S. A., et al., T cells expressing an anti-B-cell maturation antigen chimeric antigen receptor cause remissions of multiple myeloma. Blood, 2016. 128(13): p. 1688-700.
4. Seckinger, A., et al., Target Expression, Generation, Preclinical Activity, and Pharmacokinetics of the BCMA-T Cell Bispecific Antibody EM801 for Multiple Myeloma Treatment. Cancer Cell, 2017. 31(3): p. 396-410.
5. Cho, S.F., K. C. Anderson, and Y. T. Tai, Targeting B Cell Maturation Antigen (BCMA) in Multiple Myeloma: Potential Uses of BCMA-Based Immunotherapy. Front Immunol, 2018. 9: p. 1821.
6. Friedman, K. M., et al., Effective Targeting of Multiple B-Cell Maturation Antigen-Expressing Hematological Malignances by Anti-B-Cell Maturation Antigen Chimeric Antigen Receptor T Cells. Hum Gene Ther, 2018. 29(5): p. 585-601.

A number of embodiments of the invention have been described. Nevertheless, it can be understood that various modifications may be made without departing from the spirit and scope of the invention. Accordingly, other embodiments are within the scope of the following claims.

What is claimed is:

1. An isolated or purified antibody (Ab), or antigen (Ag) binding fragment thereof, or monomeric or dimeric antigen binding protein (ABP), capable of specifically binding a human B-cell maturation antigen (BCMA, or BCM) polypeptide (also called tumor necrosis factor receptor superfamily member 17 (TNFRSF17)),
wherein the isolated or purified Ab, or Ag binding fragment thereof, or monomeric or dimeric ABP comprises:
(a) a heavy chain variable region (VH) comprising:
(1) an amino acid sequence comprising the three CDR1, CDR2 and CDR3 complementarity determining regions (CDRs) of SEQ ID NO:1, or CDR1 amino acid (aa) residues GFSLKTYV (residues 25-32 of SEQ ID NO:1), CDR2 aa residues IDTGDMT (residues 50 to 56 of SEQ ID NO:1), and CDR3 aa residues ASGDI (residues 95-99 of SEQ ID NO:1), or
(2) amino acid sequences having at least about 70%, 75%, 80%, 85%, 90%, 95%, 98% sequence identity, or between about 70% to 100% sequence identity, to each of the three CDR1, CDR2 and CDR3 complementarity determining regions (CDRs) of SEQ ID NO:1, or CDR1 amino acid (aa) residues GFSLKTYV (residues 25-32 of SEQ ID NO:1), CDR2 aa residues IDTGDMT (residues 50 to 56 of SEQ ID NO:1), and CDR3 aa residues ASGDI (residues 95-99 of SEQ ID NO:1), or
(3) an amino acid sequence having at least about 70%, 75%, 80%, 85%, 90%, 95%, 98% sequence identity, or between about 70% to 100% sequence identity, to SEQ ID NO:1, or an amino acid sequence having complete sequence identity to SEQ ID NO:1;
(b) a light chain variable region (VL) comprising:
(1) an amino acid sequence comprising the three CDR1, CDR2 and CDR3 complementarity determining regions (CDRs) of SEQ ID NO:2, or CDR1 amino acid (aa) residues QSVYSNW (residues 27-33 of SEQ ID NO:2), CDR2 aa residues SAS (residues 51 to 53 of SEQ ID NO:2), and CDR3 aa residues LGEFNCARADCRA (residues 90-102 of SEQ ID NO:2), or
(2) amino acid sequences having at least about 70%, 75%, 80%, 85%, 90%, 95%, 98% sequence identity, or between about 70% to 100% sequence identity, to each of the three CDR1, CDR2 and CDR3 complementarity determining regions (CDRs) of SEQ ID NO:2, or CDR1 amino acid (aa) residues QSVYSNW (residues 27-33 of SEQ ID NO:2), CDR2 aa residues SAS (residues 51 to 53 of SEQ ID NO:2), and CDR3 aa residues LGEFNCARADCRA (residues 90-102 of SEQ ID NO:2); or
(3) an amino acid sequence having at least about 70%, 75%, 80%, 85%, 90%, 95%, 98% sequence identity, or between about 70% to 100% sequence identity, to SEQ ID NO:2, or an amino acid sequence having complete (100%) sequence identity to SEQ ID NO:2;
(c) the heavy chain variable region (VH) of (a) and the light chain variable region (VL) of (b);
(d) a heavy chain variable region (VH) comprising:
(1) an amino acid sequence comprising the three CDR1, CDR2 and CDR3 complementarity determining regions (CDRs) of SEQ ID NO:3, or CDR1 amino acid (aa) residues GFSLSAYA (residues 25-32 of SEQ ID NO:3), CDR2 aa residues INYDGIA (residues 50 to 56 of SEQ ID NO:3), and CDR3 aa residues ASDLLGVFNL (residues 93-102 of SEQ ID NO:3), or
(2) amino acid sequences having at least about 70%, 75%, 80%, 85%, 90%, 95%, 98% sequence identity, or between about 70% to 100% sequence identity, to each of the three CDR1, CDR2 and CDR3 complementarity determining regions (CDRs) of SEQ ID NO:3, or CDR1 amino acid (aa) residues GFSLSAYA (residues 25-32 of SEQ ID NO:3), CDR2 aa residues INYDGIA (residues 50 to 56 of SEQ ID NO:3), and CDR3 aa residues ASDLLGVFNL (residues 93-102 of SEQ ID NO:3), or
(3) an amino acid sequence having at least about 70%, 75%, 80%, 85%, 90%, 95%, 98% sequence identity, or between about 70% to 100% sequence identity, to SEQ ID NO:3, or an amino acid sequence having complete sequence identity to SEQ ID NO:3;
(e) a light chain variable region (VL) comprising:
(1) an amino acid sequence comprising the three CDR1, CDR2 and CDR3 complementarity determining regions (CDRs) of SEQ ID NO:4, or CDR1 amino acid (aa) residues QSVYSNNR (residues 27-34 of SEQ ID NO:4), CDR2 aa residues YAS (residues 52 to 55 of SEQ ID NO:4), and CDR3 aa residues AGGYISYSDNA (residues 91-101 of SEQ ID NO:4), or
(2) amino acid sequences having at least about 70%, 75%, 80%, 85%, 90%, 95%, 98% sequence identity, to each of the three CDR1, CDR2 and CDR3 complementarity determining regions (CDRs) of SEQ ID NO:4, or CDR1 amino acid (aa) residues QSVYSNNR (residues 27-34 of SEQ ID NO:4), CDR2 aa residues YAS (residues 52 to 55 of SEQ ID NO:4), and CDR3 aa residues AGGYISYSDNA (residues 91-101 of SEQ ID NO:4); or
(3) an amino acid sequence having at least about 70%, 75%, 80%, 85%, 90%, 95%, 98% sequence identity, or between about 70% to 100% sequence identity, to SEQ ID NO:4, or an amino acid sequence having complete (100%) sequence identity to SEQ ID NO:4;
(f) the heavy chain variable region (VH) of (d) and the light chain variable region (VL) of (e);
(g) a heavy chain variable region (VH) comprising:
(1) an amino acid sequence comprising the three CDR1, CDR2 and CDR3 complementarity determining regions (CDRs) of SEQ ID NO:5, or CDR1 amino acid (aa) residues GFSLTTYA (residues 25-32 of SEQ ID NO:5), CDR2 aa residues IWSSGTT (residues 50 to 56 of SEQ ID NO:5), and CDR3 aa residues ARYINYVTGDL (residues 93-103 of SEQ ID NO:5), or
(2) amino acid sequences having at least about 70%, 75%, 80%, 85%, 90%, 95%, 98% sequence identity, or between about 70% to 100% sequence identity, to each of the three CDR1, CDR2 and CDR3 complementarity determining regions (CDRs) of SEQ ID NO:5, or CDR1 amino acid (aa) residues GFSLTTYA (residues 25-32 of SEQ ID NO:5), CDR2 aa residues IWSSGTT (residues 50 to 56 of SEQ ID NO:5), and CDR3 aa residues ARYINYVTGDL (residues 93-103 of SEQ ID NO:5), or
(3) an amino acid sequence having at least about 70%, 75%, 80%, 85%, 90%, 95%, 98% sequence identity, or between about 70% to 100% sequence identity, to SEQ ID NO:5, or an amino acid sequence having complete sequence identity to SEQ ID NO:5;
(h) a light chain variable region (VL) comprising:
(1) an amino acid sequence comprising the three CDR1, CDR2 and CDR3 complementarity determining regions (CDRs) of SEQ ID NO:6, or CDR1 amino acid (aa) residues QSVYNNVL (residues 27-34 of SEQ ID NO:6), CDR2 aa residues KAS (residues 52 to 54 of SEQ ID NO:6), and CDR3 aa residues QGEFSCSSADCTA (residues 91-103 of SEQ ID NO:6), or
(2) amino acid sequences having at least about 70%, 75%, 80%, 85%, 90%, 95%, 98% sequence identity, to each of the three CDR1, CDR2 and CDR3 complementarity determining regions (CDRs) of SEQ ID NO:6, or CDR1 amino acid (aa) residues QSVYNNVL (residues 27-34 of SEQ ID NO:6), CDR2 aa residues KAS (residues 52 to 54 of SEQ ID NO:6), and CDR3 aa residues QGEFSCSSADCTA (residues 91-103 of SEQ ID NO:6); or
(3) an amino acid sequence having at least about 70%, 75%, 80%, 85%, 90%, 95%, 98% sequence identity, or between about 70% to 100% sequence identity, to SEQ ID NO:6, or an amino acid sequence having complete (100%) sequence identity to SEQ ID NO:6; or
(i) the heavy chain variable region (VH) of (g) and the light chain variable region (VL) of (h).

2. The isolated or purified Ab, or Ag binding fragment thereof, or monomeric or dimeric ABP, of claim 1, fabricated as or in the form of:
an antigen-binding fragment (Fab, or an Ab fragment having just one constant and one variable domain of each of an Ab heavy and light chain),
a F(ab′)₂ (or an Ab digested by pepsin yielding two fragments: a F(ab′)₂ fragment and a pFc′ (pepsin cleavage Fc) fragment),
a Fab′ (a single chain of a F(ab′)₂fragment),
a single-chain variable fragment (scFv) (or a fusion protein of a variable region of an Ab heavy and light chain connected together with a linker peptide optionally of about ten to about 25 amino acids in length),
a (scFv)₂, or a di-scFv or a bi-scFv, or a single peptide chain having two variable heavy and two variable light regions yielding tandem scFv,
a minibody (or a fusion protein of a variable region of an Ab heavy and light chain connected together with an alkyl group, optionally a methyl or an ethyl group)
a diabody (or an scFv with a linker peptide too short (optionally about five amino acids) for the two variable regions to fold together forcing the scFvs to dimerize), a triabody or a tetrabody (or an scFv with a linker peptide too short (optionally about one or two amino acids) for the two variable regions to fold together forcing the scFvs to trimerize or tetramize),
a single-domain antibody (dAB) (or a single variable region of an Ab heavy or Ab light chain),
a plurality of complementarity determining region (CDR) fragments, or
a multi specific antibody formed from two or more antibody fragments.

3. The isolated or purified Ab, or Ag binding fragment thereof, or monomeric or dimeric ABP, of any of claim 1, wherein:
(a) the heavy chain variable region (VH), if present, comprises:
(i) an amino acid sequence:
(SEQ ID NO: 1)
QSLEESRGGLFKPTDTLTLTCTVSGFSLKTYVINWVRQAP
GNGLEWIGIIDTGDMTYYASWAKSRSTITRNTNENTVTLK
MTSLTAADTATYFCASGDIWGPGTLVTVSS,
or
SEQ ID NO:1 having one or more amino acid substitutions, additions (insertions) or deletions, and the recombinant Ab, or Ag binding fragment thereof, or monomeric or dimeric ABP retains its ability to specifically bind to a human BCMA protein or polypeptide; or
(ii) an amino acid sequence:
(SEQ ID NO: 3)
QSLEESGGRLVTPGGSLTLTCTVSGFSLSAYAMSWVRQAP
GKGLEWIGYINYDGIAYYTNWAKGRFTISKTSTTVDLKIT
SPTTEDTAAYFCASDLLGVFNLWGQGTLVTVSS,
or
SEQ ID NO:3. having one or more amino acid substitutions, additions (insertions) or deletions, and the recombinant Ab, or Ag binding fragment thereof, or monomeric or dimeric ABP retains its ability to specifically bind to a human BCMA protein or polypeptide; or
(iii) an amino acid sequence:
(SEQ ID NO: 5)
QSVEESGGRLVTPGTPLTLTCTASGFSLTTYAMGWVRQAP
GKGLEWIGYIWSSGTTDYASWAKGRFTISKTSPTVDLKMT
SPTTEDTATYFCARYINYVTGDLWGQGTLVTVSS,
or
SEQ ID NO:5. having one or more amino acid substitutions, additions (insertions) or deletions, and the recombinant Ab, or Ag binding fragment thereof, or monomeric or dimeric ABP retains its ability to specifically bind to a human BCMA protein or polypeptide;
and optionally the one or more amino acid substitutions comprise one or more conservative amino acid substitutions;
(b) the light chain variable region (VL), if present, comprises:
(i) an amino acid sequence:
(SEQ ID NO: 2)
AQVLTQTASPVSAAVGGTVTINCQASQSVYSNWLSWFQQK
PGQPPKRLIYSASTLASGVSSRFKGSGSGTQFTLTISDVQ
CDDAATYYCLGEFNCARADCRAFGGGTEVVVK,
or
SEQ ID NO:2 having one or more amino acid substitutions, additions (insertions) or deletions, and the recombinant Ab, or Ag binding fragment thereof, or monomeric or dimeric ABP retains its ability to specifically bind to a human BCMA protein or polypeptide; or
(ii) an amino acid sequence:
(SEQ ID NO: 4)
AAVLTQTASPVSAAVGGTVTINCQASQSVYSNNRLSWFQQ
KPGQPPKLLIYYASFLASGVPSRFKGSGSGAQFTLTISDL
QCDDAATYYCAGGYISYSDNAFGGGTEVVVK,
or
SEQ ID NO:4 having one or more amino acid substitutions, additions (insertions) or deletions, and the recombinant Ab, or Ag binding fragment thereof, or monomeric or dimeric ABP, retains its ability to specifically bind to a human BCMA protein or polypeptide; or
(iii) an amino acid sequence:
(SEQ ID NO: 6)
AQVLTQTPSPVSADVGGTVTINCQASQSVYNNVLLAWYQQ
KAGQPPKLLIYKASTLASGVSSRFKGSGSGTQFTLTISGV
QCDDAATYYCQGEFSCSSADCTAFGGGTEVVVK,
or
SEQ ID NO:6 having one or more amino acid substitutions, additions (insertions) or deletions, and the recombinant Ab, or Ag binding fragment thereof, or monomeric or dimeric ABP, retains its ability to specifically bind to a human BCMA protein or polypeptide;
and optionally the one or more amino acid substitutions comprise one or more conservative amino acid substitutions, and the isolated or purified Ab, or Ag binding fragment thereof, or monomeric or dimeric ABP, retains its ability to specifically bind to a human BCMA protein or polypeptide;
and optionally SEQ ID NO:1, SEQ ID NO:2, SEQ ID NO:3 or SEQ ID NO:4 has two, three, four, five, six, seven, eight, nine, ten, eleven, twelve thirteen, fourteen or fifteen conservative amino acid substitutions, and the recombinant Ab, or Ag binding fragment thereof, or monomeric or dimeric ABP retains its ability to specifically bind to a human BCMA protein or polypeptide; or
(c) the light chain variable region further comprises at least a portion of a light chain constant region,
and optionally the light chain constant region comprises an amino acid sequence:
(SEQ ID NO: 7)
GDPVAPTVLIFPPAADQVATGTVTIVCVANKYFPDVTVTW
EVDGTTQTTGIENSKTPQNSADCTYNLSSTLTLTSTQYNS
HKEYTCKVTQGTTSVVQSFNRGDC;
or
(d) the heavy chain variable region further comprises at least a portion of a heavy chain constant region
and optionally the heavy chain constant region comprises an amino acid sequence:
(SEQ ID NO: 8)
GQPKAPSVFPLAPCCGDTPSSTVTLGCLVKGYLPEPVTVT
WNSGTLTNGVRTFPSVRQSSGLYSLSSVVSVTSSSQPVTC
NVAHPATNTKVDKTVAPSTCSKPTCPPPELLGGPSVFIFP
PKPKDTLMISRTPEVTCVVVDVSQDDPEVQFTWYINNEQV
RTARPPLREQQFNSTIRVVSTLPIAHQDWLRGKEFKCKVH
NKALPAPIEKTISKARGQPLEPKVYTMGPPREELSSRSVS
LTCMINGFYPSDISVEWEKNGKAEDNYKTTPAVLDSDGSY
FLYSKLSVPTSEWQRGDVFTCSVMHEALHNHYTQKSISRS
PGK;
or
(e) the light chain variable region further comprises at least a portion of a light chain constant region; and, the heavy chain variable region further comprises at least a portion of a heavy chain constant region;
and optionally the heavy chain constant region comprises amino acid sequence from a IgG, IgM, IgA, IgD or IgE isotype; or
(f) the light chain constant region comprises amino acid sequence from a kappa (κ) or lambda (λ) isotype; or
(g) the at least a portion of the heavy chain constant region, at least a portion of the light chain constant region, or at least a portion of the heavy chain constant region and the light chain constant region, is or comprises amino acid sequence of a human, a rabbit, a mouse or a rat origin or comprises constant region amino acid sequence derived from a human, a rabbit, a mouse or a rat; or
(h) at least a portion of the heavy chain constant region, at least a portion of the light chain constant region, or at least a portion of the heavy chain constant region and the light chain constant region, is or comprises a synthetic amino acid sequence; or
(i) the recombinant Ab, the Ag binding fragment thereof, or monomeric or dimeric ABP, or the heavy chain constant region, or the light chain constant region, or the heavy chain constant region and the light chain constant region, further comprises or is bound to a heterologous protein, peptide, or a compound or a composition,
and optionally the heterologous protein or peptide, or the compound or a composition, comprises a detectable protein, a detectable agent or a binding moiety,
and optionally the heterologous protein or peptide comprises a carrier protein,
and optionally the heterologous protein, peptide or the compound or composition, is covalently conjugated to the recombinant antibody (Ab), or Ag binding fragment thereof, or monomeric or dimeric ABP,
and optionally the detectable agent or binding moiety comprises a biotin, a fluorescent or chemiluminescent label, a fluorophore, perylene, fluorenyl, coumarin, 7-methoxycoumarin (Mca), 4-(dimethylaminoazo)benzene-4-carboxylic acid (dabcyl), Tamra, boron-dipyrromethene (BODIPY), or derivatives thereof, a dye, a radioisotope, a quantum dot or photoluminescent aqueous nanocrystal, a hapten, or an antibody binding epitope or domain,
and optionally the dye is or comprises rhodamine, [2-(4-nitro-2,1,3-benzoxadiazol-7-yl)aminoethyl]trimethylammonium (NBD), nile red or nile blue, or is a fluorescent dye comprising sulfoindocyanine,and optionally the fluorophore is or comprises a dansyl, a fluorescein, a carboxyfluorescein (FAM) or a 6-FAM moiety,
and optionally the dye is or comprises a cyanine dye, a Cy3 or a Cy5,
and optionally the hapten is or comprises a biotin, a theophylline, a digoxigenin, a carborane, a fluorescein or a bromodeoxyuridine moiety: and/or
(j) the Ab, or Ag binding fragment thereof, or monomeric or dimeric ABP is a recombinant Ab, or Ag binding fragment thereof, or monomeric or dimeric ABP, or comprises a peptide or polypeptide made by a recombinant technique.

4. A chimeric or recombinant nucleic acid comprising: a nucleic acid sequence encoding an Ab, or Ag binding fragment thereof, or monomeric or dimeric ABP of claim 1,
and optionally the chimeric or recombinant nucleic acid further comprises and is operatively linked to a transcriptional regulatory element, and optionally the transcriptional regulatory element comprises a promoter, and optionally the promoter is an inducible promoter or a constitutive promoter,
and optionally the chimeric or recombinant nucleic acid further comprises sequence encoding an amino terminal signal peptide,
and optionally the amino terminal signal peptide comprises the amino acid sequence:
(SEQ ID NO: 9)
METGLRWLLLVAVLKGVQC;
(SEQ ID NO: 10)
MDTRAPTQLLGLLLLWLPGATF;
(SEQ ID NO: 11)
METGLRWLLLVAVLKGVQC;
(SEQ ID NO: 12)
MDTRAPTQLLGLLLLWLPGAAF;
(SEQ ID NO: 13)
METGLRWLLLVAVLKGVQC;
or
(SEQ ID NO: 14)
MDTRAPTQLLGLLLLWLPGATF,
and optionally the amino terminal signal peptide SEQ ID NO:9 is amino terminal to VH SEQ ID NO:1; the amino terminal signal peptide SEQ ID NO:10 is amino terminal to VL SEQ ID NO:2; the amino terminal signal peptide SEQ ID NO:11 is amino terminal to VH SEQ ID NO:3; the amino terminal signal peptide to VL SEQ ID NO:12 is amino terminal to SEQ ID NO:4; the amino terminal signal peptide to VH SEQ ID NO:13 is amino terminal to SEQ ID NO:5, and the amino terminal signal peptide to VL SEQ ID NO:14 is amino terminal to SEQ ID NO:6.

5. An expression cassette, a vector, a recombinant virus, an artificial chromosome, a cosmid or a plasmid comprising a chimeric or a recombinant nucleic acid of claim 4.

6. A cell comprising a chimeric or recombinant antibody or dimeric antigen binding protein of claim 1,
and optionally the cell is a bacterial, fungal, mammalian, yeast, insect or plant cell, or a lymphocyte, or a T cell,
and optionally the cell expresses or secretes the chimeric or recombinant antibody or dimeric antigen binding protein extracellularly or into the extracellular milieu, or at least the antigen binding moiety of the chimeric or recombinant antibody or dimeric antigen binding protein cell is expressed or displayed extracellularly or into the extracellular milieu,
and optionally the mammalian cell is a human cell, or a human lymphocyte, or a human T cell.

7. A method for detecting the presence of a human B-cell maturation antigen (BCMA, or BCM) protein or polypeptide in or on a cell, a tissue, an organ or a portion of any of the foregoing, comprising:
(a) contacting the cell, tissue or organ or portion of any of the foregoing with at least one Ab, or Ag binding fragment thereof, or monomeric or dimeric ABP of any of claim 1, and
(b) detecting the specific binding of the at least one Ab, or Ag binding fragment thereof, or monomeric or dimeric ABP with a BCMA polypeptide, in or on the cell, tissue or organ or portion of any of the foregoing,
thereby detecting the presence of the human BCMA protein in or on the cell, tissue, organ or portion of any of the foregoing,
wherein optionally the at least one Ab, or Ag binding fragment thereof, or monomeric or dimeric ABP comprises a variable heavy chain (VH) having an amino acid sequence comprising SEQ ID NO:1 and a variable light chain (VL) having an amino acid sequence comprising SEQ ID NO:2,
and optionally the at least one Ab, or Ag binding fragment thereof, or monomeric or dimeric ABP comprises a variable heavy chain (VH) having an amino acid sequence comprising SEQ ID NO:3 and a variable light chain (VL) having an amino acid sequence comprising SEQ ID NO:4,
and optionally the at least one Ab, or Ag binding fragment thereof, or monomeric or dimeric ABP comprises a variable heavy chain (VH) having an amino acid sequence comprising SEQ ID NO:5 and a variable light chain (VL) having an amino acid sequence comprising SEQ ID NO:6,
and optionally the method comprises contacting the cell, tissue or organ or portion of any of the foregoing with two Abs, or Ag binding fragments thereof, or monomeric or dimeric ABPs, or a mixture of two Abs, or Ag binding fragments thereof, or monomeric or dimeric ABPs,
and optionally one of the two Abs, or Ag binding fragments thereof, or monomeric or dimeric ABPs comprises a variable heavy chain (VH) having an amino acid sequence comprising SEQ ID NO:1 and a variable light chain (VL) having an amino acid sequence comprising SEQ ID NO:2; and the second of the two Abs, or Ag binding fragments thereof, or monomeric or dimeric ABPs comprises a variable heavy chain (VH) having an amino acid sequence comprising SEQ ID NO:3 and a variable light chain (VL) having an amino acid sequence comprising SEQ ID NO:4,
and optionally one of the two Abs, or Ag binding fragments thereof, or monomeric or dimeric ABPs comprises a variable heavy chain (VH) having an amino acid sequence comprising SEQ ID NO:3 and a variable light chain (VL) having an amino acid sequence comprising SEQ ID NO:4; and the second of the two Abs, or Ag binding fragments thereof, or monomeric or dimeric ABPs comprises a variable heavy chain (VH) having an amino acid sequence comprising SEQ ID NO:5 and a variable light chain (VL) having an amino acid sequence comprising SEQ ID NO:6,
and optionally one of the two Abs, or Ag binding fragments thereof, or monomeric or dimeric ABPs comprises a variable heavy chain (VH) having an amino acid sequence comprising SEQ ID NO:1 and a variable light chain (VL) having an amino acid sequence comprising SEQ ID NO:2; and the second of the two Abs, or Ag binding fragments thereof, or monomeric or dimeric ABPs comprises a variable heavy chain (VH) having an amino acid sequence comprising SEQ ID NO:5 and a variable light chain (VL) having an amino acid sequence comprising SEQ ID NO:6,
and optionally the contacting comprises use of an immunohistochemistry (IHC) assay,
and optionally the method further comprises contacting the Ab, or Ag binding fragment thereof, or monomeric or dimeric ABP, specifically bound to a BCMA polypeptide, with a detectable agent to indicate or signal the specific binding of the Ab, or Ag binding fragment thereof, or monomeric or dimeric ABP, to the human BCMA protein,
and optionally the detectable agent specifically binds to the Ab, or Ag binding fragment thereof, or monomeric or dimeric ABP,
and optionally the cell, tissue, organ or a portion of any of the foregoing is or comprises: a lymphocyte, and optionally the lymphocyte comprises or is a B-cell or B lymphocyte, and optionally the B-cell or B lymphocyte comprises or is an early B cell, a pro-B cell, a pre-B lymphocyte, a mature B-lymphocyte, and optionally the lymphocyte comprises or is a T-cell or T lymphocyte, and optionally the T-cell or T lymphocyte comprises or is a parafollicular T lymphocyte or a subpopulation of parafollicular T lymphocytes,
and optionally the cell, tissue, organ or a portion of any of the foregoing is or comprises: a cancer or malignant cell, and optionally the cancer or malignant cell comprises or is a breast myoepithelial cell, a leukemia cell, a carcinoma cell, a carcinoid tumor cell, a mamma carcinoma cell, a colon carcinoma cell, a malignant melanoma cell, a multiple myeloma cell, a plasmacytoma cell or a lymphoma cell,
and optionally the cell, tissue, organ or a portion of any of the foregoing is or comprises: a follicular center cell, or a cell in a tonsil, an organ, a lymph node germinal center, a bone marrow stem cell, a myelopoietic cell, a liver bile canalicular cell, a renal glomerular cell, a proximal tubular cell, a stromal cell around or associated with an infiltrating tumor cell, a kidney cell, an epithelial cell, a cerebellum cell, a prostate cell, a kidney cell, a pancreas cell, or a bone marrow cell,
and optionally the epithelial cell is or is derived from a brain, lung, intestine, kidney, breast or placental epithelial cell,′
and optionally the organ is a liver, prostate or lung,
and optionally the leukemia cell is an acute lymphoblastic leukemia (ALL) cell,
and optionally the carcinoma cell is a basal cell carcinoma (BCC) cell,
and optionally the lymphoma is a B cell lymphoma, and optionally the B cell lymphoma is a Hodgkin's lymphoma or a non-Hodgkin's lymphoma, and optionally the non-Hodgkin's lymphoma is follicular lymphoma, a diffuse large B cell lymphoma, a marginal zone B cell lymphoma, a small lymphocytic lymphoma (SLL), a chronic lymphocytic leukemia, (CLL), a Burkitt lymphoma or a mantle cell lymphoma (MCL).

8. A method for detecting or diagnosing a cancer,
wherein the method comprises detecting expression or presence of a human BCMA protein or peptide in or on a cell, tissue or organ sample using a method of claim 7,
wherein the detecting of the specific binding of the Ab, or Ag binding fragment thereof, or monomeric or dimeric ABP with a BCMA polypeptide in or on the cell, tissue or organ or portion of any of the foregoing, detects or diagnoses, or assists in the detection or diagnosis of, the cancer,
wherein optionally the cancer is: leukemic cell cancer of pre-B phenotype, a lymphoma, a plasmocytoma, chronic myelogenous leukemia in blast crisis, diffuse large B-cell lymphoma, hairy cell leukemia, a myeloma, precursor T lymphoblastic leukemia or lymphoma, a non-hematolymphoid sarcoma, or a carcinoma,
and optionally the lymphoma is a B cell lymphoma, a precursor B lymphoblastic leukemia or lymphoma, a follicular lymphoma, an acute lymphocytic leukemia (ALL), an angioimmunoblastic T cell lymphoma, a Burkitt's lymphoma, a diffuse large B-cell lymphoma, a mantle cell lymphoma or an angioimmunoblastic T-cell lymphoma, and optionally the B cell lymphoma is a Hodgkin's lymphoma or a non-Hodgkin's lymphoma, and optionally the non-Hodgkin's lymphoma is follicular lymphoma, a diffuse large B cell lymphoma, a marginal zone B cell lymphoma, a small lymphocytic lymphoma (SLL) or chronic lymphocytic leukemia, (CLL), or a mantle cell lymphoma (MCL),
and optionally the carcinoma is a renal cell carcinoma or a metaplastic breast carcinoma,
and optionally the cell, tissue or organ sample is from an individual in need thereof,
and optionally the detection comprises conducting an immunohistochemistry (IHC) assay,
and optionally at least two Abs, or Ag binding fragments thereof, or monomeric or dimeric ABPs, are used to contact the cell, tissue or organ sample,
and optionally one of the two Abs, or Ag binding fragments thereof, or monomeric or dimeric ABPs comprises a variable heavy chain (VH) having an amino acid sequence comprising SEQ ID NO:3 and a variable light chain (VL) having an amino acid sequence comprising SEQ ID NO:4; and the second of the two Abs, or Ag binding fragments thereof, or monomeric or dimeric ABPs comprises a variable heavy chain (VH) having an amino acid sequence comprising SEQ ID NO:5 and a variable light chain (VL) having an amino acid sequence comprising SEQ ID NO:6.

9. A method for treating, ameliorating or preventing a cancer comprising first detecting or diagnosing the cancer using a method of claim 8, followed by treatment of the individual in need thereof for the treatment, amelioration or prevention of the cancer,
wherein optionally the cancer is: leukemic cell cancer of pre-B phenotype, a lymphoma, chronic myelogenous leukemia in blast crisis, diffuse large B-cell lymphoma, hairy cell leukemia, a myeloma, a precursor B lymphoblastic leukemia or lymphoma, a follicular lymphoma, precursor T lymphoblastic leukemia or lymphoma, a non-hematolymphoid sarcoma, or a carcinoma,
and optionally the lymphoma is a B cell lymphoma, acute lymphocytic leukemia (ALL), angioimmunoblastic T cell lymphoma, a Burkitt's lymphoma, diffuse large B-cell lymphoma, mantle cell lymphoma, a plasmocytoma, angioimmunoblastic T-cell lymphoma,
and optionally the B cell lymphoma is a Hodgkin's lymphoma or a non-Hodgkin's lymphoma, and optionally the non-Hodgkin's lymphoma is follicular lymphoma, a diffuse large B cell lymphoma, a marginal zone B cell lymphoma, a small lymphocytic lymphoma (SLL) or chronic lymphocytic leukemia, (CLL), or a mantle cell lymphoma (MCL),
and optionally the carcinoma is a renal cell carcinoma or a metaplastic breast carcinoma.

10. A kit comprising: a chimeric or recombinant antibody of claim 1,
wherein optionally the kit comprises components needed for an immunohistochemistry (IHC) assay.

11. A kit comprising: a recombinant nucleic acid of claim 4,
wherein optionally the kit comprises components needed for an immunohistochemistry (IHC) assay.

12. A kit comprising: a cell of claim 6,
wherein optionally the kit comprises components needed for an immunohistochemistry (IHC) assay.
